(19) United States
(12) Patent Application Publication
(10) Pub. No.: US 2024/0285268 A1
(43) Pub. Date: Aug. 29, 2024

(54) TISSUE COLLECTOR WITH SHUTTLE AND EXPANDABLE SHAFT

(71) Applicant: GYRUS ACMI, INC. D/B/A OLYMPUS SURGICAL TECHNOLOGIES AMERICA, Westborough, MA (US)

(72) Inventors: Nikhil M. Murdeshwar, Maple Grove, MN (US); Thomas J. Holman, Princeton, MN (US)

(21) Appl. No.: 18/570,735

(22) PCT Filed: Jun. 22, 2022
(86) PCT No.: PCT/US2022/073078

§ 371 Date: Dec. 15, 2023

Related U.S. Application Data
(60) Provisional application No. 63/213,847, filed on Jun. 23, 2021.

(51) Int. Cl. A61B 10/04; A61B 10/06

(52) U.S. Cl. CPC A61B 10/04; A61B 10/06

(57) ABSTRACT

A tissue collection instrument comprises a tissue separator device comprising a separator and a storage volume, and a tissue retrieval device comprises a tissue shuttle storable in the storage volume and a retriever connected to the tissue shuttle to retract the tissue shuttle away from the tissue separator device. A method of collecting biological matter using a tissue collection instrument comprises inserting a tissue separator device into anatomy. operating the tissue separator device to obtain a first tissue sample from the anatomy. collecting the first tissue sample with a shuttle associated with the tissue separator device and withdrawing the shuttle from the anatomy. A method of collecting biological matter comprises assembling a tissue separator with a scope, inserting the scope and the tissue separator into anatomy. operating the tissue separator to obtain a tissue sample. and withdrawing the tissue sample through an openable lumen in the scope.

### PRIORITY CLAIM

This application claims the benefit of priority to U.S. Provisional Patent Application Ser. No. 63/213,847, filed Jun. 23, 2021, the contents of which are incorporated herein by reference.

### TECHNICAL FIELD

The present disclosure relates generally to medical devices comprising elongate bodies configured to be inserted into incisions or openings in anatomy of a patient to provide diagnostic or treatment operations.

More specifically, the present disclosure relates to medical devices that can be inserted into anatomy of a patient to perform a biological matter removal process, such as by cutting sample tissue for analysis.

### BACKGROUND

Endoscopes can be used for one or more of 1) providing passage of other devices, e.g., therapeutic devices or tissue collection devices, toward various anatomical portions, and 2) imaging of such anatomical portions. Such anatomical portions can include gastrointestinal tract (e.g., esophagus, stomach, duodenum, pancreaticobiliary duct, intestines, colon, and the like), renal area (e.g., kidney(s), ureter, bladder, urethra) and other internal organs (e.g., reproductive systems, sinus cavities, submucosal regions, respiratory tract), and the like.

Conventional endoscopes can be involved in a variety of clinical procedures, including, for example, illuminating, imaging, detecting and diagnosing one or more disease states, providing fluid delivery (e.g., saline or other preparations via a fluid channel) toward an anatomical region, providing passage (e.g., via a working channel) of one or more therapeutic devices for sampling or treating an anatomical region, and providing suction passageways for collecting fluids (e.g., saline or other preparations) and the like.

In conventional endoscopy, the distal portion of the endoscope can be configured for supporting and orienting a therapeutic device, such as with the use of an elevator. In some systems, two endoscopes can be configured to work together with a first endoscope guiding a second endoscope inserted therein with the aid of the elevator. Such systems can be helpful in guiding endoscopes to anatomic locations within the body that are difficult to reach. For example, some anatomic locations can only be accessed with an endoscope after insertion through a circuitous path.

### SUMMARY

The present inventors have recognized that problems to be solved with conventional medical devices, and in particular endoscopes and duodenoscopes used to retrieve sample biological matter, include, among other things, 1) the difficulty in navigating endoscopes, and instruments inserted therein, to locations in anatomical regions deep within a patient, 2) the disadvantage of only being able to obtain small tissue sample sizes 3) the increased time and associated cost of having to repeatedly remove and reinsert medical devices to obtain a sufficient quantity of sample material, and 4), the difficulty of incorporating features (e.g., steerability and tissue collection features) into small-diameter devices, particularly without obstructing optical devices (e.g., imaging and lighting components) mounted to the endoscope. Such problems can be particularly present in duodenoscopy procedures (e.g., Endoscopic Retrograde Cholangio-Pancreatography, hereinafter “ERCP” procedures) where an auxiliary scope (also referred to as daughter scope, or cholangioscope) can be attached and advanced through the working channel of a “main scope” (also referred to as mother scope or duodenoscope). Furthermore, tissue collection and retrieval devices used to remove the sample matter can be inserted through the auxiliary scope. As such, the duodenoscope, auxiliary scope and tissue retrieval device become progressively smaller and more difficult to maneuver and perform interventions and treatments.

The present disclosure can help provide solutions to these and other problems by providing systems, devices and methods relating to inserting tissue retrieval devices, such as biopsy forceps, with an auxiliary scope having a small-diameter passage. The tissue retrieval devices can be tethered or otherwise attached to a distal end of an endoscope to allow the tissue retrieval device to be sized beyond the constraints of the lumen of the endoscope. The tissue retrieval device can thereby have increased capacity for storing obtained sample tissue, thereby reducing or eliminating the need to remove the endoscope to empty the tissue retrieval device for another sample collection insertion iteration.

Furthermore, in order to facilitate navigation of the endoscope with the tissue retrieval device located distally thereof and the tissue collection process, the tissue retrieval device can be optically enhanced, such as by being made of translucent or clear materials to allow visibility of optical devices through and into the tissue retrieval device. Other optically enhanced materials can include reflective materials to allow for interaction of the material with light to improve recognition by the optical device. Optically enhanced tissue retrieval devices can be configured to bend light waves, such as to provide optical magnification. The optically enhanced material can allow for viewing of: 1) target tissue to be collected by the tissue retrieval device, 2) tissue inside the tissue retrieval device, 3) newly exposed tissue after some target tissue has been separated from the anatomy, and 4) components of the tissue retrieval device relative to the target tissue, as well as other benefits.

Additionally, owing in part to being freed of the size constraints of the endoscope lumen, the tissue retrieval device can include features to facilitate obtaining multiple samples of tissue without previously collected samples becoming dislodged from (e.g., falling out of) the tissue retrieval device and to increase the holding capacity of the tissue retrieval device. Thus, the tissue retrieval devices can be configured to hold one or more pieces of sample material, thereby allowing collection of multiple samples and larger samples in a single insertion pass.

In additional aspects, the present disclosure can provide the ability retrieve multiple tissue samples from the patient with only having to insert the relevant tissue retrieval device one time. For example, the tissue retrieval device can include a tissue collection shuttle that can be pulled by a retriever out of the tissue retrieval device along with a first tissue sample to allow the tissue retrieval device to collect a second tissue sample. In variations, the tissue collection shuttle can be pulled through an openable lumen within a scope that can expand in cross-sectional area, such as by opening flaps or flanges, to allow a tissue collection shuttle filled with tissue to be pulled through the scope even if the tissue collection shuttle is larger than the nominal cross-sectional area of the openable lumen. In additional examples, an expandable tissue retrieval device can be pulled through an openable lumen in the scope.

As such, the present disclosure can help solve the problems referenced above and other problems by 1) reducing the number of times a tissue retrieval device needs to be inserted and reinserted into the anatomy, 2) increasing the capacity of sample material collected with each insertion, and 3) increasing the number of samples that can be collected, among other things, as is described herein. Such solutions can be achieved by A) locating distally of an endoscope a tissue retrieval device that can be larger than the lumen of the endoscope to increase size, B) providing optically enhanced tissue retrieval devices to reduce or eliminate interference with imaging capabilities, C) providing tissue collection shuttles that can withdraw a first tissue sample from anatomy independent of the tissue retrieval device, D) providing expandable or enlargeable tissue retrieval devices and tissue collection shuttles, and E) providing openable lumens on shafts of scopes to allow for enlarged tissue retrieval devices and shuttles to pass therethrough.

The terms “tissue retrieval device,” “tissue retriever,” “tissue collection device,” “tissue collector” and the like can refer to instruments configured to be inserted into anatomy and bring back a tissue sample upon withdrawal. Such devices do not necessarily perform any actual separation of tissue from the anatomy and can thus collect and retrieve tissue already or previously separated. The term “tissue separation device” and “tissue separator” can refer to an instrument specifically configured to separate tissue, such as by cutting, punching, sawing, tearing and the like. Thus, a tissue separation device does not necessarily collect or retrieve tissue but can do so in various examples. Likewise, a tissue retrieval or collection device can perform tissue separation in various examples. A biopsy instrument can comprise a tissue separation device (e.g., forceps or jaws) configured to collect (e.g., hold separated tissue between the jaws) and retrieve (e.g., withdraw) the collected tissue from the anatomy. A tissue retrieval device can encompass a tissue shuttle, as described herein, which can collect and retrieve tissue, but not necessarily separate tissue. The term “tissue” can refer to “biological matter” and the like.

In an example, a tissue collection instrument can comprise a tissue separator device comprising a separator and a storage volume, and a tissue retrieval device comprising a tissue shuttle configured to be storable in the storage volume and a retriever connected to the tissue shuttle to retract the tissue shuttle away from the tissue separator device.

In another example, a method of collecting biological matter using a tissue collection instrument can comprise inserting a tissue separator device into anatomy, operating the tissue separator device to obtain a first tissue sample from the anatomy, collecting the first tissue sample with a shuttle associated with the tissue separator device and withdrawing the shuttle from the anatomy.

In an additional example, a working shaft of a medical scope device can comprise an elongate body extending from a first end portion to a second end portion, an imaging component connected to the elongate body, a working channel extending at least partially through the elongate body between the first end portion and the second end portion, and a tissue retrieval channel extending at least partially through the elongate body between the first end portion and the second end portion, the tissue retrieval channel comprising an enlargeable cross-sectional area openable to an exterior of the elongate body.

In another example, a method of collecting biological matter can comprise assembling a tissue separator device with a scope, inserting the scope and the tissue separator device into anatomy, operating the tissue separator device to obtain a first tissue sample from the anatomy, and withdrawing the first tissue sample through an openable lumen in the scope.

### BRIEF DESCRIPTION OF DRAWINGS

FIG. 1 is a schematic diagram of an endoscopy system comprising an imaging and control system and an endoscope, such as duodenoscope, with which the biological matter collection systems and devices of the present disclosure can be used.

FIG. 2 is a schematic diagram of the imaging and control system of FIG. 1 showing the imaging and control system connected to the endoscope.

FIG. 3A is a schematic top view of a distal portion of the endoscope of FIG. 2 comprising a camera module including optical components for a side-viewing endoscope and an elevator mechanism.

FIG. 3B is an enlarged cross-sectional view taken along the plane 3B-3B of FIG. 3A showing the optical components.

FIG. 3C is an enlarged cross-sectional view taken along the plane 3C-3C of FIG. 3A showing the elevator mechanism.

FIG. 4 is a schematic illustration of a distal portion of an endoscope being used to position an auxiliary scope proximate a duodenum, the auxiliary scope being configured to receive a tissue retrieval device, including a tethered biopsy instrument, of the present disclosure.

FIG. 5A is a schematic illustration of a tissue retrieval device of the present disclosure comprising an elongate shaft and a translucent tissue collector.

FIG. 5B is a close-up view of a distal end of the tissue retrieval device of FIG. 5A showing the translucent tissue collector disposed within an auxiliary endoscope.

FIG. 6A is a schematic illustration of a translucent tissue collector comprising forceps in a closed state.

FIG. 6B is a schematic illustration of the translucent tissue collector of FIG. 6A with the forceps in an open state.

FIG. 7A is a schematic illustration of a translucent tissue collector comprising a boring device extending from an endoscope having an imaging device.

FIG. 7B is a schematic cross-sectional illustration of the translucent tissue collector of FIG. 7A with collected tissue inside the translucent tissue collector.

FIG. 8A is a schematic illustration of an endoscopy system comprising an endoscope and a tethered biopsy instrument.

FIG. 8B is a side view of a forceps suitable for use as the biopsy instrument of FIG. 8A.

FIG. 9 is a schematic illustration of a biopsy instrument comprising forceps having a tissue retention system comprising a sponge and needles.

FIG. 10 is schematic illustration of a biopsy instrument comprising forceps having expandable jaws.

FIG. 11 is a schematic illustration of a biopsy instrument comprising forceps having a flexible jaw.

FIG. 12 is a block diagram illustrating methods of collecting biological matter from a patient using tethered biopsy instruments of the present disclosure.

FIG. 13 is perspective view of a scope having a tissue separator and a tissue collection shuttle connected thereto.

FIG. 14 is a perspective view of the tissue collection shuttle of FIG. 13.

FIG. 15A is a schematic side view of a tissue collection shuttle suitable for use with the scope and tissue separator of FIG. 13 and disposed inside of a pair of jaws.

FIG. 15B is a schematic side view of the tissue collection shuttle of FIG. 15A with the tissue collection shuttle retracted from the pair of jaws.

FIG. 16 is a schematic side view of a tissue collection shuttle suitable for use with the scope and tissue separator of FIG. 13 comprising a corkscrew.

FIG. 17 is schematic perspective view of a scope comprising a shaft having an openable lumen and a tissue collection shuttle located in a tissue separator device.

FIG. 18 is a schematic cross-sectional view of the shaft of FIG. 17 wherein the openable lumen is unobstructed by the tissue collection shuttle such that retainer flanges are in an opposed state.

FIG. 19 is a schematic perspective view of the scope of FIG. 17 wherein the tissue collection shuttle is located within the openable lumen.

FIG. 20 is a schematic cross-sectional view of the shaft of FIG. 19 wherein the openable lumen includes the tissue collection shuttle such that retainer flanges are turned out.

FIG. 21 is a block diagram illustrating methods of collecting biological matter from a patient using a tissue collection shuttle and an openable lumen.

### DETAILED DESCRIPTION

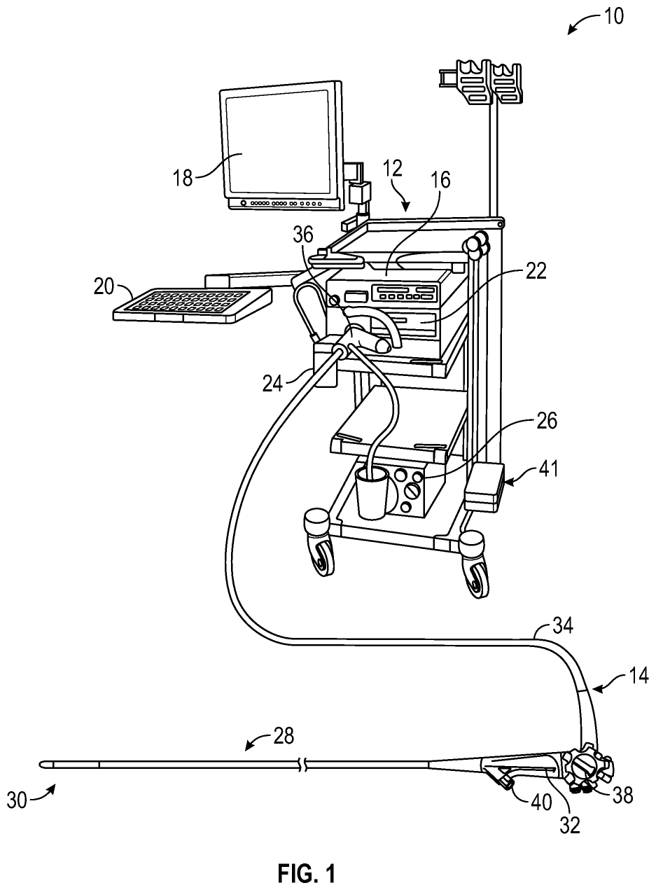

FIG. 1 is a schematic diagram of endoscopy system 10 comprising imaging and control system 12 and endoscope 14. The system of FIG. 1 is an illustrative example of an endoscopy system suitable for use with the systems, devices and methods described herein, such as tethered and optically enhanced biological matter and tissue collection, retrieval and storage devices and biopsy instruments that can be used for obtaining samples of tissue or other biological matter to be removed from a patient for analysis or treatment of the patient. According to some examples, endoscope 14 can be insertable into an anatomical region for imaging and/or to provide passage of or attachment to (e.g., via tethering) one or more sampling devices for biopsies, or one or more therapeutic devices for treatment of a disease state associated with the anatomical region. Endoscope 14 can, in advantageous aspects, interface with and connect to imaging and control system 12. In the illustrated example, endoscope 14 comprises a duodenoscope, though other types of endoscopes can be used with the features and teachings of the present disclosure.

Imaging and control system 12 can comprise control unit 16, output unit 18, input unit 20, light source 22, fluid source 24 and suction pump 26.

Imaging and control system 12 can include various ports for coupling with endoscopy system 10. For example, control unit 16 can include a data input/output port for receiving data from and communicating data to endoscope 14. Light source 22 can include an output port for transmitting light to endoscope 14, such as via a fiber optic link. Fluid source 24 can include a port for transmitting fluid to endoscope 14. Fluid source 24 can comprise a pump and a tank of fluid or can be connected to an external tank, vessel or storage unit. Suction pump 26 can comprise a port used to draw a vacuum from endoscope 14 to generate suction, such as for withdrawing fluid from the anatomical region into which endoscope 14 is inserted. Output unit 18 and input unit 20 can be used by an operator of endoscopy system 10 to control functions of endoscopy system 10 and view output of endoscope 14. Control unit 16 can additionally be used to generate signals or other outputs from treating the anatomical region into which endoscope 14 is inserted. In examples, control unit 16 can generate electrical output, acoustic output, a fluid output and the like for treating the anatomical region with, for example, cauterizing, cutting, freezing and the like.

Endoscope 14 can comprise insertion section 28, functional section 30 and handle section 32, which can be coupled to cable section 34 and coupler section 36.

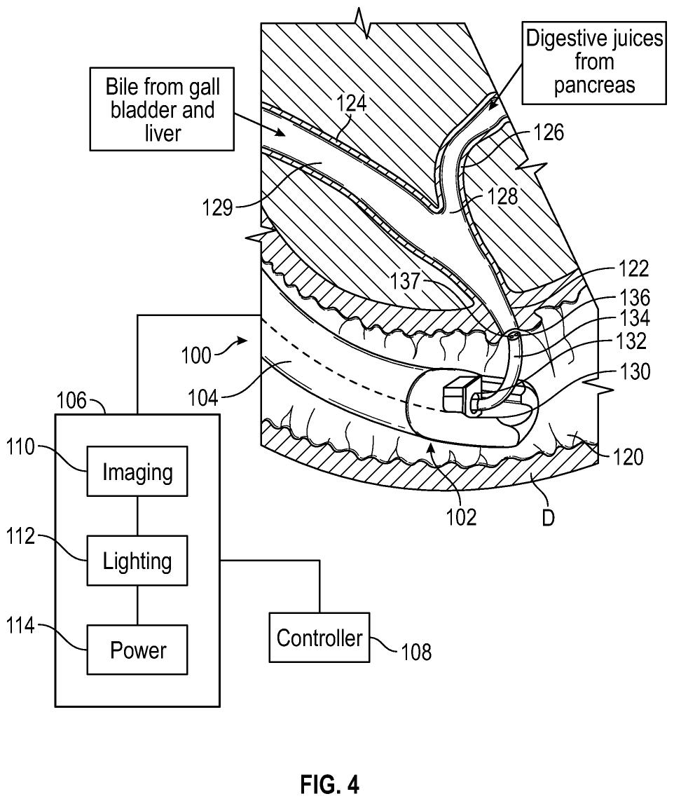

Insertion section 28 can extend distally from handle section 32 and cable section 34 can extend proximally from handle section 32. Insertion section 28 can be elongate and include a bending section, and a distal end to which functional section 30 can be attached. The bending section can be controllable (e.g., by control knob 38 on handle section 32) to maneuver the distal end through tortuous anatomical passageways (e.g., stomach, duodenum, kidney, ureter, etc.). Insertion section 28 can also include one or more working channels (e.g., an internal lumen) that can be elongate and support insertion of one or more therapeutic tools of functional section 30, such as auxiliary scope 134 of FIG. 4. The working channel can extend between handle section 32 and functional section 30. Additional functionalities, such as fluid passages, guide wires, and pull wires can also be provided by insertion section 28 (e.g., via suction or irrigation passageways, and the like).

Handle section 32 can comprise knob 38 as well as ports 40. Knob 38 can be coupled to a pull wire, or other actuation mechanisms, extending through insertion section 28. Ports 40 can be configured to couple various electrical cables, guide wires, auxiliary scopes, tissue collection devices of the present disclosure, fluid tubes and the like to handle section 32 for coupling with insertion section 28.

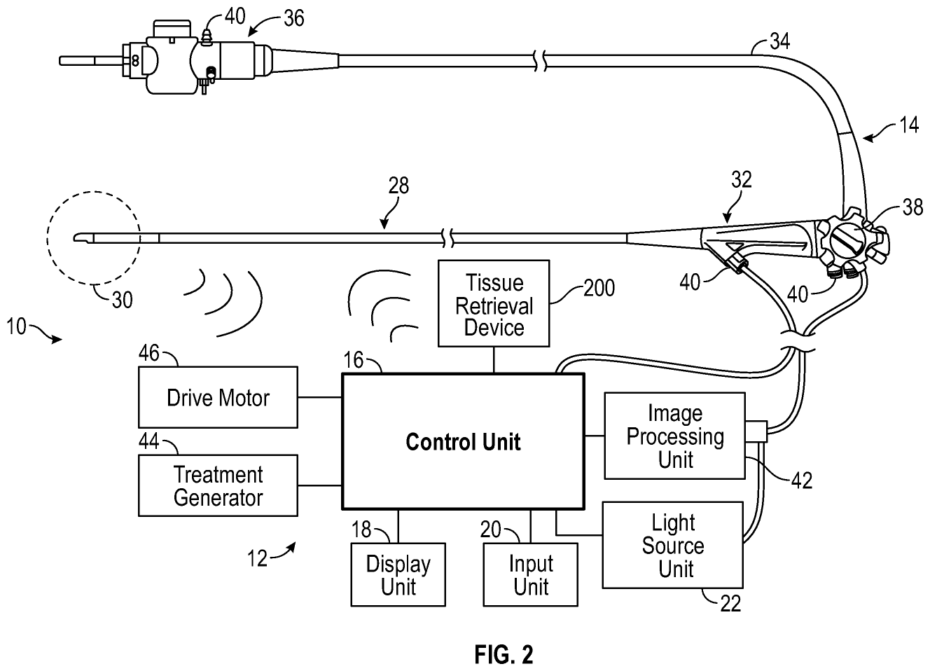

Imaging and control system 12, according to examples, can be provided on a mobile platform (e.g., cart 41) with shelves for housing light source 22, suction pump 26, image processing unit 42 (FIG. 2), etc. Alternatively, several components of imaging and control system 12 shown in FIGS. 1 and 2 can be provided directly on endoscope 14 so as to make the endoscope “self-contained.”

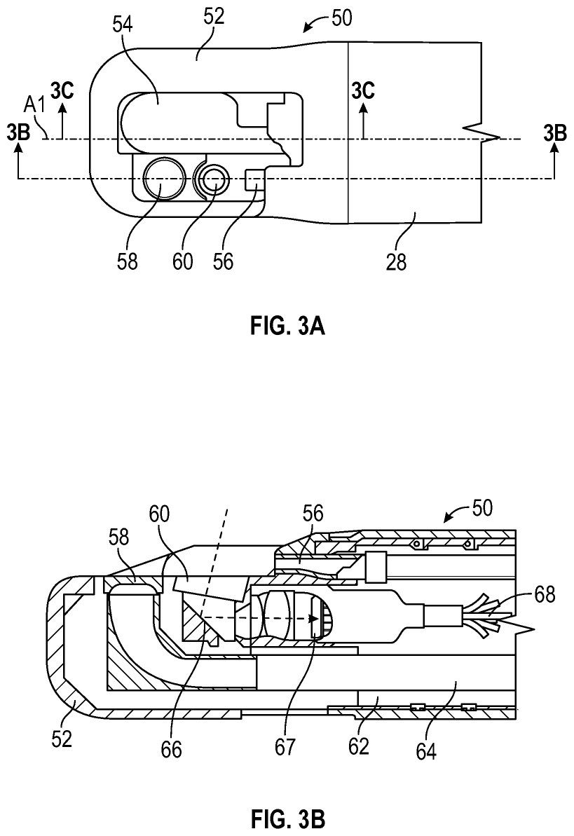

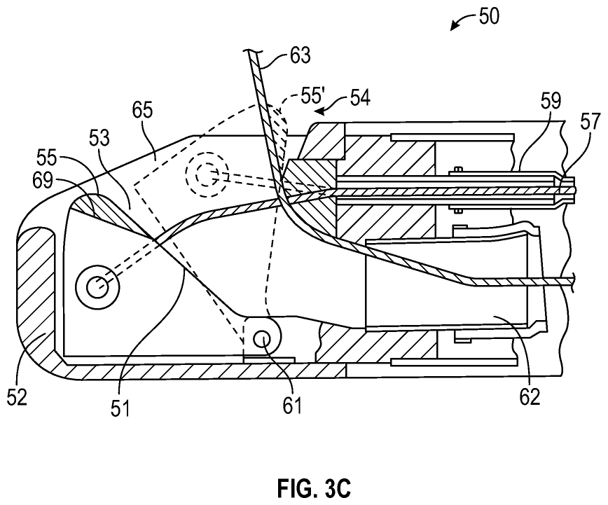

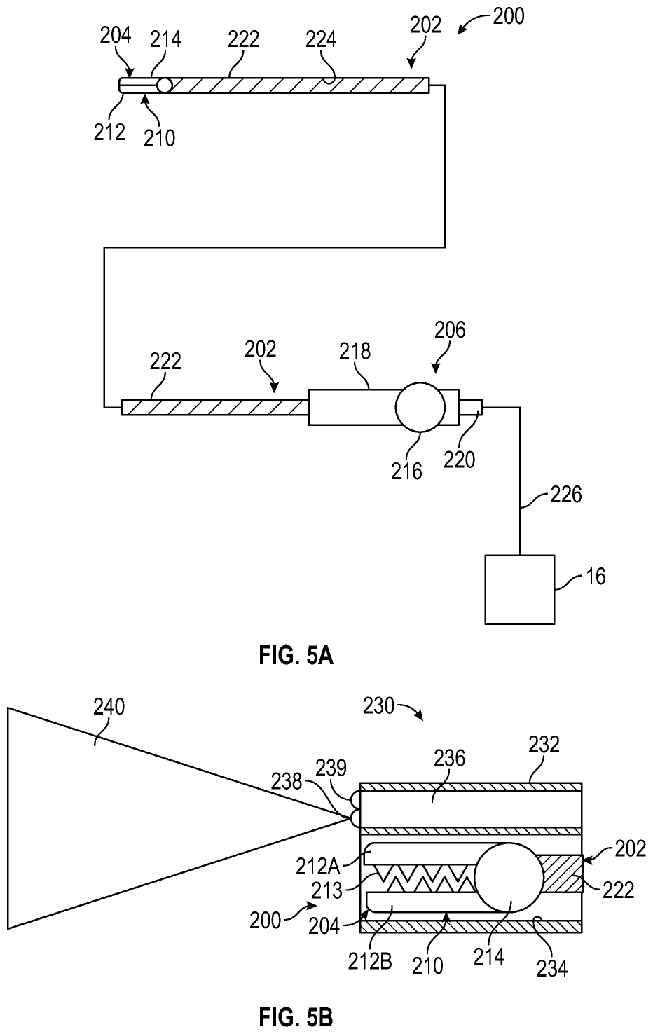

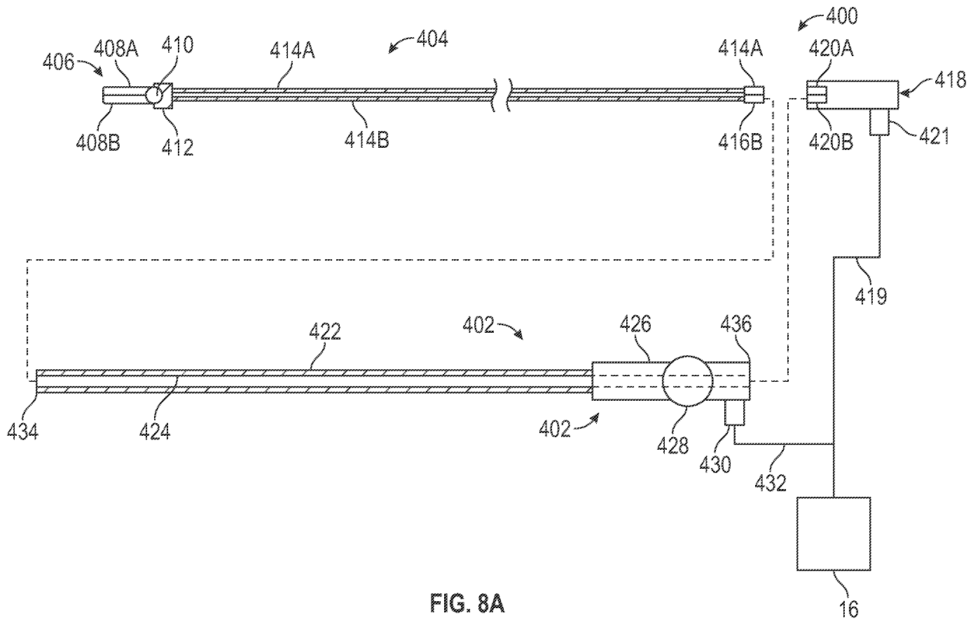

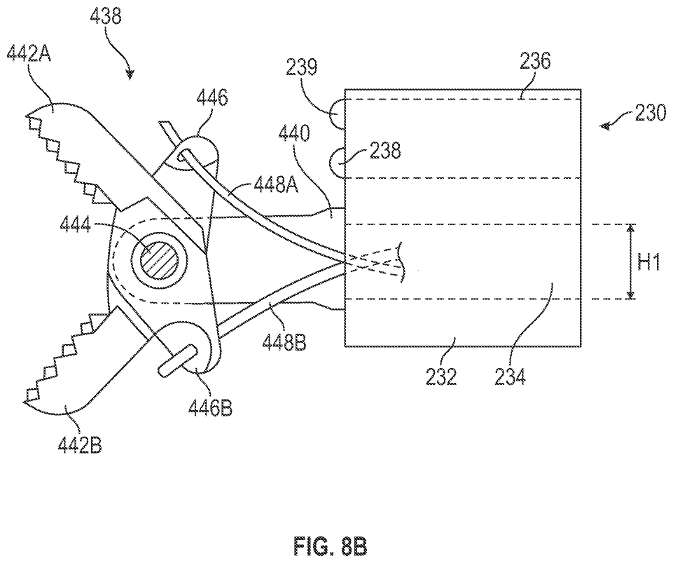

Functional section 30 can comprise components for treating and diagnosing anatomy of a patient. Functional section 30 can comprise an imaging device, an illumination device and an elevator, such as is described further with reference to elevator 54 of FIGS. 3A-3C. Functional section 30 can further comprise optically enhanced biological matter and tissue collection and retrieval devices as are described herein. For example, functional section 30 can comprise one or more electrodes conductively connected to handle section 32 and functionally connected to imaging and control system 12 to analyze biological matter in contact with the electrodes based on comparative biological data stored in imaging and control system 12. In other examples, functional section 30 can directly incorporate tissue collectors similar to the tissue retrieval devices described with reference to FIGS. 5A-7B and the biopsy devices described with reference to FIGS. 8A-11.

FIG. 2 is a schematic diagram of endoscopy system 10 of FIG. 1 comprising imaging and control system 12 and endoscope 14. FIG. 2 schematically illustrates components of imaging and control system 12 coupled to endoscope 14, which in the illustrated example comprises a duodenoscope. Imaging and control system 12 can comprise control unit 16, which can include or be coupled to image processing unit 42, treatment generator 44 and drive unit 46, as well as light source 22, input unit 20 and output unit 18. As is discussed below in greater detail with reference to FIGS. 4-5B, control unit 16 can comprise, or can be in communication with, endoscope 100, surgical instrument 200 and endoscopy system 400, which can comprise a device configured to engage tissue and collect and store a portion of that tissue and through which imaging equipment (e.g., a camera) can view target tissue via inclusion of optically enhanced materials and components. Control unit 16 can be configured to activate a camera to view target tissue distal of surgical instrument 200 and endoscopy system 400, which can be fabricated of a translucent material to minimize the impacts of the camera being obstructed or partially obstructed by the tissue retrieval device. Likewise, control unit 16 can be configured to activate light source unit 22 to shine light on surgical instrument 200, which can include select components that are configured to reflect light in a particular manner, such as tissue cutters being enhanced with reflective particles.

Image processing unit 42 and light source 22 can each interface with endoscope 14 (e.g., at functional unit 30) by wired or wireless electrical connections. Imaging and control system 12 can accordingly illuminate an anatomical region, collect signals representing the anatomical region, process signals representing the anatomical region, and display images representing the anatomical region on display unit 18. Imaging and control system 12 can include light source 22 to illuminate the anatomical region using light of desired spectrum (e.g., broadband white light, narrow-band imaging using preferred electromagnetic wavelengths, and the like). Imaging and control system 12 can connect (e.g., via an endoscope connector) to endoscope 14 for signal transmission (e.g., light output from light source, video signals from imaging system in the distal end, diagnostic and sensor signals from a diagnostic device, and the like).

Fluid source 24 (FIG. 1) can be in communication with control unit 16 and can comprise one or more sources of air, saline or other fluids, as well as associated fluid pathways (e.g., air channels, irrigation channels, suction channels) and connectors (barb fittings, fluid seals, valves and the like). Fluid source 24 can be utilized as an activation energy for a biasing device or a pressure-applying device of the present disclosure. Imaging and control system 12 can also include drive unit 46, which can be an optional component. Drive unit 46 can comprise a motorized drive for advancing a distal section of endoscope 14, as described in at least PCT Pub. No. WO 2011/140118 A1 to Frassica et al., titled “Rotate-to-Advance Catheterization System,” which is hereby incorporated in its entirety by this reference.

FIGS. 3A-3C illustrate a first example of functional section 30 of endoscope 14 of FIG. 2. FIG. 3A illustrates a top view of functional section 30. FIG. 3B illustrates a cross-sectional view of functional section 30 taken along section plane 3B-3B of FIG. 3A. FIG. 3C illustrates a cross-sectional view of functional section 30 taken along section plane 3C-3C of FIG. 3A. FIGS. 3A-3C illustrate “side-viewing endoscope” (e.g., duodenoscope) camera module 50. In side-viewing endoscope camera module 50, illumination and imaging systems are positioned such that the viewing angle of the imaging system corresponds to a target anatomy lateral to central longitudinal axis Al of endoscope 14. However, the biological matter retrieval devices can be used with other types of endoscopes, such as “end-viewing endoscopes.”

In the example of FIGS. 3A and 3B, side-viewing endoscope camera module 50 can comprise housing 52, elevator 54, fluid outlet 56, illumination lens 58 and objective lens 60. Housing 52 can form a fluid tight coupling with insertion section 28. Housing 52 can comprise opening for elevator 54. Elevator 54 can comprise a mechanism for moving a device inserted through insertion section 28, such as auxiliary scope 134 of FIG. 4. In particular, elevator 54 can comprise a device that can bend an elongate device extended through insertion section 28 along axis Al, as is discussed in greater detail with reference to FIG. 3C. Elevator 54 can be used to bend the elongate device at an angle to axis Al to thereby treat or access the anatomical region adjacent side-viewing endoscope camera module 50. Elevator 54 is located alongside, e.g., radially outward of axis Al, illumination lens 58 and objective lens 60.

As can be seen in FIG. 3B, insertion section 28 can comprise central lumen 62 through which various components (e.g., auxiliary scope 134 (FIG. 4) can be extended to connect functional section 30 with handle section 32 (FIG. 2). For example, illumination lens 58 can be connected to light transmitter 64, which can comprise a fiber optic cable or cable bundle extending to light source 22 (FIG. 1). Likewise, objective lens 60 can be coupled to prism 66 and imaging unit 67, which can be coupled to wiring 68. Also, fluid outlet 56 can be coupled to fluid line 69, which can comprise a tube extending to fluid source 24 (FIG. 1). Other elongate elements, e.g., tubes, wires, cables, can extend through lumen 62 to connect functional section 30 with components of endoscopy system 10, such as suction pump 26 (FIG. 1) and treatment generator 44 (FIG. 2).

FIG. 3C a schematic cross-sectional view taken along section plane 3C-3C of FIG. 30 showing elevator 54. Elevator 54 can comprise deflector 55 that can be disposed in space 53 of housing 52. Deflector 55 can be connected to wire 57, which can extend through tube 59 to connect to handle section 32. Wire 57 can be actuated, such as by rotating a knob, pulling a lever, or pushing a button on handle section 32. Movement of wire 57 can cause rotation, e.g., clockwise, from a first position of deflector 55 about pin 61 to a second position of deflector 55, indicated by 55′. Deflector 55 can be actuated by wire 57 to move the distal portion of instrument 63 extending through window 65 in housing 52.

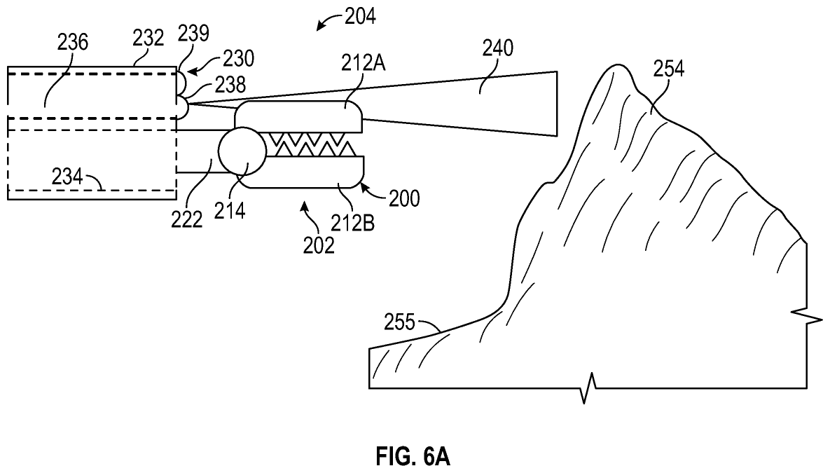

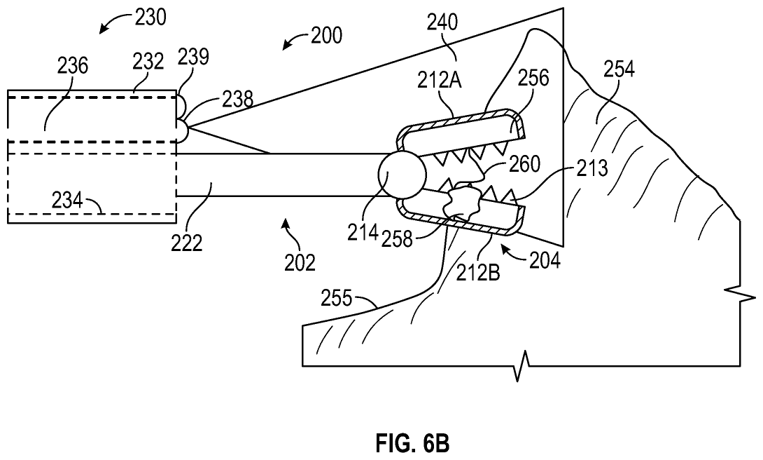

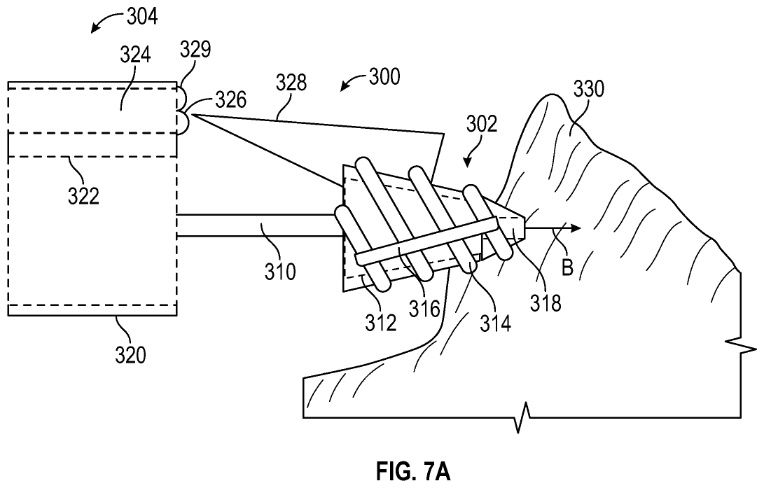

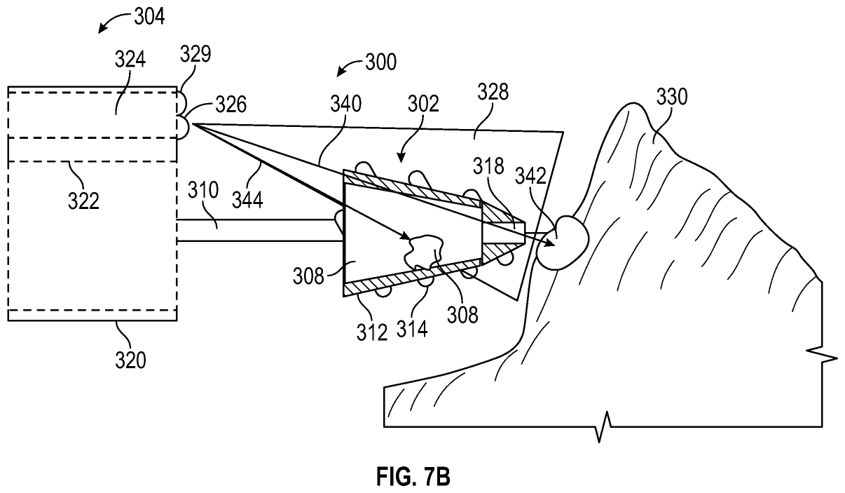

Housing 52 can comprise accommodation space 53 that houses deflector 55. Instrument 63 can comprise forceps, a guide wire, a catheter, or the like that extends through lumen 62. Instrument 63 can additionally comprise auxiliary scope 134 of FIG. 4, or a tissue collection device such as surgical instrument 200 of FIGS. 5A-6B and tissue retrieval device 300 (FIG. 7A), as well as other instruments such as biopsy instruments 404 of FIG. 8A. A proximal end of deflector 55 can be attached to housing 62 at pin 61 provided to the rigid tip 21. A distal end of deflector 55 can be located below window 65 within housing 62 when deflector 55 is in the lowered, or un-actuated, state. The distal end of deflector 55 can at least partially extend out of window 65 when deflector 55 is raised, or actuated, by wire 57. Instrument 63 can slide on angled ramp surface 51 of deflector 55 to initially deflect the distal end of instrument 63 toward window 65. Angled ramp surface 51 can facilitate extension of the distal portion of instrument 63 extending from window 65 at a first angle relative to the axis of lumen 62. Angled ramp surface 51 can include groove 69, e.g. a v-notch, to receive and guide instrument 63. Deflector 55 can be actuated to bend instrument 63 at a second angle relative to the axis of lumen 62, which is closer to perpendicular that the first angle. When wire 57 is released, deflector 55 can be rotated, e.g., counter-clockwise, back to the lowered position, either by pushing or relaxing of wire 57. In examples, instrument 63 can comprise a cholangioscope or auxiliary scope 134 (FIG. 4).

Side-viewing endoscope camera module 50 of FIGS. 3A-3C can include optical components (e.g., objective lens 60, prism 66, imaging unit 67, wiring 68) for collection of image signals, lighting components (e.g., illumination lens 58, light transmitter 64) for transmission or generation of light. Endoscope camera module 50 can also include a photosensitive element, such as a charge-coupled device (“CCD” sensor) or a complementary metal-oxide semiconductor (“CMOS”) sensor. In either example, imaging unit 67 can be coupled (e.g., via wired or wireless connections) to image processing unit 42 (FIG. 2) to transmit signals from the photosensitive element representing images (e.g., video signals) to image processing unit 42, in turn to be displayed on a display such as output unit 18. In various examples, imaging and control system 12 and image processing unit 67 can be configured to provide outputs at desired resolution (e.g., at least 480p, at least 720p, at least 1080p, at least 4K UHD, etc.) suitable for endoscopy procedures.

Thus, as endoscope 100 is inserted further into the anatomy, the complexity with which it must be maneuvered and contorted increases, as described with reference to FIG. 4. Furthermore, in order to reach locations even further in the anatomy, additional devices can be used, e.g., instrument 63 in the form of auxiliary scope 134. As such, the cross-sectional area, e.g., diameter, of subsequently nested devices becomes smaller, thereby requiring even smaller devices that can be difficult to manufacture and manipulate, or satisfactorily produce results without repeated interventions (e.g., interactions with the patient), as is described with reference to FIGS. 5A-7B.

FIG. 4 is a schematic illustration of distal portion of endoscope 100 according to the present disclosure positioned in duodenum D. Endoscope 100 can comprise functional module 102, insertion section module 104, and control module 106. Control module 106 can include controller 108. Control module 106 can include other components, such as those described with reference to endoscopy system 10 (FIG. 1) and control unit 16 (FIG. 2). Additionally, control module 106 can comprise components for controlling a camera and a light source connected to auxiliary scope 134, such as imaging unit 110, lighting 112 and power unit 114. Endoscope 100 can be configured similarly as endoscope 14 of FIGS. 1 and 2.

Duodenum D can comprise duct wall 120, sphincter of Oddi 122, common bile duct 124 and main pancreatic duct 126. Duodenum D comprises an upper part of the small intestine. Common bile duct 124 carries bile from the gallbladder and liver (not illustrated) and empties the bile into the duodenum D through sphincter of Oddi 122. Main pancreatic duct 126 carries pancreatic juice from the exocrine pancreas (not illustrated) to common bile duct 124. Sometimes it can be desirable to remove biological matter, e.g., tissue, from bile duct 124 or pancreatic duct 126 to analyze the tissue to, for example, diagnose diseases or maladies of the patient such as cancer.

Functional module 102 can comprise elevator portion 130. Endoscope 100 can further comprise lumen 132 and auxiliary scope 134. Auxiliary scope 134 can comprise lumen 136. Auxiliary scope 134 can itself include functional components, such as camera lens 137 and a light lens (not illustrated) coupled to control module 106, to facilitate navigation of auxiliary scope 134 from endoscope 100 through the anatomy and to facilitate viewing of components extending from lumen 132.

In certain duodenoscopy procedures (e.g., Endoscopic Retrograde Cholangio-Pancreatography, hereinafter “ERCP” procedures) an auxiliary scope (also referred to as daughter scope, or cholangioscope), such as auxiliary scope 134, can be attached and advanced through lumen 132 (or central lumen 62 of insertion section 28 of endoscope 14 in FIG. 3B) of the “main scope” (also referred to as mother scope, or duodenoscope), such as endoscope 100. As discussed in greater detail below, auxiliary scope 134 can be guided into sphincter of Oddi 122. Therefrom, a surgeon operating auxiliary scope 134 can navigate auxiliary scope 134 through lumen 132 toward the gall bladder, liver or other locations in the gastrointestinal system to perform various procedures. The surgeon can navigate auxiliary scope 134 past entry 128 of main pancreatic duct 126 and into passage 129 of common bile duct 124, or into entry 128. Auxiliary scope 134 can be used to guide an additional device to the anatomy to obtain biological matter, such as by passage through or attachment to lumen 136. The additional device can have its own functional devices, such as a light source, camera, tissue separators, accessories, and biopsy channel, for therapeutic procedures. As described with reference to FIGS. 5A-7B, the additional device can include various features, such as forceps or an auger, for gathering biological matter, such as tissue. As described with reference to FIGS. 8A-11, the additional device can comprise a biopsy device tethered to the endoscope and that has tissue collection capacity enhancement features. The biological matter can then be removed from the patient, typically by removal of the additional device from the auxiliary device, so that the removed biological matter can be analyzed to diagnose one or more conditions of the patient. According to several examples, endoscope 100 can be suitable for the removal of cancerous or pre-cancerous matter (e.g., carcinoma, sarcoma, myeloma, leukemia, lymphoma and the like), endometriosis evaluation, biliary ductal biopsies, and the like.

However, as mentioned above, the size of the additional device is typically small due to the progressively smaller sizes of endoscope 100, auxiliary scope 134 and the additional device. In examples, lumen 132 of endoscope 100 can typically be on the order of approximately 4.0 mm in diameter, while lumen 136 of auxiliary scope 134 can typically be on the order of approximately 1.2 mm. As such, with conventional devices, it can be difficult to obtain sufficiently large tissue sample sized to ensure accurate diagnoses without having to repeatedly remove and reinsert the additional device. Likewise, it can be difficult to view the desired matter, e.g., the target tissue, due to multiple reasons including the presence of the tissue retrieval device in the line of sight of the auxiliary scope camera. This thereby makes collection of non-desirable, e.g., non-cancerous, material a possibility. However, with the systems and devices of the present disclosure it is possible to obtain sufficiently large tissue sample sizes with only a single insertion and removal of the additional device, when configured as a tissue retrieval device or biopsy instrument of the present disclosure, for example. For example, the tissue retrieval device can be fabricated partially or entirely of translucent materials to allow imaging devices to have improved visibility of tissue behind the tissue retrieval device. Additionally, the tissue retrieval device can be fabricated partially or entirely of reflective materials to allow imaging devices to have improved visibility of particular components, e.g., functional components such as tissue cutters, of the tissue retrieval device. Furthermore, the present disclosure include tissue retrieval devices and biopsy devices that can be placed out front of the auxiliary scope and the lumen extending therethrough to increase the size and capacity of the tissue collection device.

FIG. 5A is a schematic illustration of surgical instrument 200 comprising elongate body 202, tissue collection device 204 and device controller 206. Surgical instrument 200 can comprise a device configured for the separation, collection and retrieval of biological matter, such as tissue, from a patient. Tissue collection device 204 can comprise separator 210, which, in the illustrated example, comprises jaws 212 and hinge 214 and activation mechanism 216. Controller 206 can comprise handpiece or handle 218, which can include activation mechanism 216 and connector 220. Elongate body 202 can comprise shaft 222 that can include lumen 224. Controller 206 can be connected to system control unit 16 (FIGS. 1 and 2) via cable 226 and the use of connector 220. The components illustrated in FIGS. 5A and 5B are not necessarily drawn to scale.

Tissue collection device 204 can be configured to do one or both of separate and retrieve biological matter from within a patient after being positioned within the patient by elongate body 202. Tissue collection device 204 can be configured to engage target tissue, separate the target tissue from the patient and store separated target tissue for removal from the patient, such as by removal of elongate body 202 from the patient.

Handpiece 218 can comprise any device suitable for facilitating manipulation and operation of surgical instrument 200. Handpiece 218 can be located at the proximal end of shaft 222 or another suitable location along shaft 222. In examples, handpiece 218 can comprise a pistol grip, a knob, a handlebar grip and the like. Actuation mechanism 216 can be attached to handpiece 218 to operate tissue collection device 204. Actuation mechanism 216 can comprise one or more of buttons, triggers, levers, knobs, dials and the like. Actuation mechanism 216 can be coupled to pressure-applying device 214 and can comprise any suitable device for allowing operation of pressure-applying device 214 from handpiece 218. As such, actuation mechanism 216 can comprise a linkage located within lumen 224 of shaft 222 or alongside shaft 222. In examples, the linkage can be a mechanical linkage, an electronic linkage or an electric linkage, (such as a wire or cable), or an activation energy source, such as an electric source, a fluid source or a gas source (such as a tube or conduit).

Shaft 222 can extend from handpiece 218 and can comprise an elongate member configured to allow tissue collection device 204 to be inserted into a patient. In examples, shaft 222 can be sized for placement within an auxiliary scope, such as scope 134 of FIG. 4. As such, shaft 222 can be inserted into an incision in the epidermis of a patient, through a body cavity of the patient and into an organ. Thus, it is desirable for the diameter or cross-sectional shape of shaft 222, as well as components attached thereto, to be as small as possible to facilitate minimally invasive surgical procedures. Tissue collection device 204 can thus be incorporated into shaft 222 to minimize the size impact on surgical instrument 200 and without interfering with the linkage. Shaft 222 can be axially rigid, but resiliently bendable, and formed from a metal or plastic material.

Tissue collection device 204 can be located at the distal end of shaft 222 or another suitable location along shaft 222. Tissue collection device 204 can be sized to fit within lumen 136 (FIG. 4), for example. Tissue collection device 204 can comprise a component or device for interacting with a patient, such as those configured to cut, slice, pull, saw, punch, twist or auger tissue, and the like. Specifically, separator 210 can comprise any device suitable for removing tissue from a patient, such as a blade, punch, scraping device or an auger. In additional examples, separator 212 can comprise a device configured to scrape or abrade tissue from the patient, such as a brush or grater device. In another example, separator 212 can comprise a roughened surface, such as a surface coated with hard particles, such as diamond or sand particles. Separator 210 can be configured to physically separate portions of tissue of a patient from other larger portions of tissue in the patient. In additional examples, separator 210 can be configured to simply collect biological matter from the patient that does not need physical separation, such as mucus or fluid. In examples, separator 212 can be configured to physically separate portion of tissue of a patient for retrieval with the tissue collection device or another device. In the illustrated example, separator 210 can comprise forceps having jaws 212 pivotably connected at hinge 214. Separator 210 can, however, be configured as a variety of devices capable of collecting biological matter, such as a punch, an auger, a blade, a saw and the like. Likewise, separator 210 can incorporate features for storing collected matter, such as a container or storage space. In an example, as discussed with reference to FIG. 6A, the storage space can be provided between jaws. Separator 210 can comprise forceps, as is described with reference to FIGS. 6A and 6B, or an auger, as is described with reference to FIGS. 7A and 7B. In any configuration, portions of separator 210 can be configured to allow light to pass therethrough or to reflect light incident thereon to selectively enhance images of separator 210 and anatomy obtained by an imaging unit.

Jaws 212 can be configured as a container or a walled element to hold and retain biological matter collected by tissue collection device 204. In an example, jaws 212 can comprise a flexible basket that can be deformed to allow portions of jaws 212 to be brought into close contact with target tissue. For example, jaws 212 can be fabricated from woven material such as strands of Kevlar, PVC, polyethylene, polycarbonate, PEEK and the like. Jaws 212 can be coupled to structural components, e.g., a frame, to facilitate coupling to shaft 222 and to facilitate mounting of cutting elements, such as teeth or blades, to jaws 212, as well as to provide stability for separator 210. In additional examples, jaws 212 can comprise a structural element, such as a box fabricated from rigid and inflexible material.

Handpiece 218 can be operated by a user to operate tissue collection device 204. Handpiece 218 can be used to manipulate shaft 222 to push separator 210 against target tissue. For example, shaft 222 can be rotated, oscillated, reciprocated and the like move separator 210 along the target tissue to cause separator 210 to separate sample tissue from the target tissue attached to the patient. Activation mechanism 216 can be coupled to handpiece 218 and can be configured to operate separator 210. Activation mechanism 216 can comprise any type of device suitable for activating the different types of separator devices described herein. In examples, activation mechanism 216 can comprise one or more of a lever, a trigger, a joystick, a button, a wheel and the like, as well as combinations thereof. In an example, activation mechanism 216 can comprise a wheel that can be rotated in one direction to open jaws 212 and rotated in an opposite direction to close jaws 212. For example, the wheel can be rotated to push and/or pull a wire to open and close jaws 212.

FIG. 5B is a close-up view of a distal end of tissue collection device 204 of FIG. 5A showing translucent tissue separator 210 extending from auxiliary endoscope 230. Endoscope 230 can comprise an example of auxiliary scope 134. Endoscope 230 can comprise shaft 232, working channel 234, passage 236 and lens 238. Field of view 240 can project from lens 238. Endoscope 230 can additionally include lens 239 for the projection of light into field of view 240.

Tissue collection device 204 can be configured as a low-profile device so as to be able to be inserted through a small diameter lumen, such as lumen 136 of auxiliary scope 134 of FIG. 4. Additionally, tissue collection device 204 can be configured as a high-capacity tissue collector that can hold a large volume of collected sample tissue to thereby reduce or eliminate the need to repeatedly remove surgical instrument 200 from the auxiliary scope. Furthermore, tissue collection device 204 can be optically enhanced to facilitate user operation of tissue collection device 204 to interact with target tissue. For example, jaws 212A and 212B can be fabricated from translucent material to allow lens 238 to see through jaws 212A and 212B, and teeth 213 can be fabricated of reflective material to reflect light from lens 239 back to lens 238 to allow a user to more clearly delineate where tissue collection device 204 will interact with target tissue of the patient. Jaws 212A and 212B can further be configured to provide magnification of target tissue when viewed through one or both of jaws 212A and 212B. In examples, one or both of jaws 212A and 212B can include one or more convex surfaces of transparent material to provide optical magnification.

Tissue collection device 204 can be fully retracted into working channel 234. Working channel 234 can comprise lumen 136 of FIG. 4. As such, lens 238 can be freely moved by manipulation of shaft 232 to position target tissue within field of view 240. However, when it is desired to extend tissue collection device 204 from working channel 234, tissue collection device 204 can become positioned within field of view 240, thereby inhibiting or preventing lens 238 from capturing images of the target tissue. As discussed with reference to FIGS. 6A-7B, tissue collection device 204 can be configured to allow light to 1) pass through components, portions or all of collector 210, and/or 2) be reflected by components, portions or all of collector 210 to enhance images obtained through lens 238.

FIG. 6A is a schematic illustration of surgical instrument 200 wherein separator device 210 comprises forceps 250 in a closed state and extended from endoscope 230 proximate target tissue 254. FIG. 6B is a schematic illustration of surgical instrument 200 with separator device 210 in a deployed state with forceps 250 open to engage target tissue 254. FIGS. 6A and 6B are discussed concurrently and the components therein are not necessarily drawn to scale.

As shown in FIG. 6A, tissue collection device 204 can be positioned in an anatomic duct 255 where target tissue 254 is located. Shaft 222 can be used to guide separator 210 through an anatomic duct to target tissue 254. Target tissue 254 can comprise a protrusion, such as a growth of cancerous or pre-cancerous material.

Endoscope 230 can be positioned such that lens 238 faces target tissue 254. As such, target tissue 254 can be within field of view 240 of lens 238. Field of view 240 is illustrated as having a particular viewing angle. However, lens 238 can be configured to have field of view 240 with different angles, up to and including one-hundred-eight degrees. As can be seen in FIG. 6A, tissue collection device 204 extended from shaft 232 to expose jaws 212A and 212B, but to not yet engage target tissue 254. As such, jaws 212A and 212B can thus be located to not completely block field of view 240 from target tissue 254. However, field of view 240 can become obstructed the further tissue collection device 204 becomes extended from working channel 234. For example, the portion of duct 255 from which target tissue 254 extends can become blocked from viewing by lens 238.

FIG. 6B is a side view of tissue collection device 204 with jaws 212 shown in cross-section to show storage space 256 with sample tissue 258. Jaws 212 can be elongated in the radial directions (e.g., up and down with respect to the orientations of FIG. 6B) so as to form a container for the storage of collected matter.

With jaws 212 rotated away from each other at hinge 214, tissue collection device 204 can be moved in the axial direction toward sample tissue 258. Jaws 212 can be rotated toward each other to engage target tissue 254. Tissue collection device 204 can be reciprocated back-and-forth along the axis of shaft 222 to collect sample tissue 258. Teeth 213 can be used to cut, saw, tear or rip portions of target tissue 254 away from the anatomy of the patient. In examples, only one of jaws 212A and 212B can be configured to rotate.

Teeth 213 can be fabricated out of an edge of jaws 212A and 212B. In examples, teeth 213 can comprise extensions of the material of jaws 212A and 212B. In such examples, both teeth 213 and jaws 212A and 212B can be fabricated of a rigid material such as plastic or metal. In examples, jaws 212A and 212B can be fabricated from Gorilla Glass® commercially available from Corning, or other chemically strengthened glass such as alkali-aluminosilicate sheet glass. In examples, jaws 212A and 212B can be fabricated from molded polycarbonate.

In additional examples, teeth 213 and jaws 212A and 212B can be mounted to a frame extending from hinge 214. For example, jaw 212A can comprise a U-shaped, rigid frame having end portions extending from hinge 214 to form a bounded space. Jaw 212A can comprise a bag or bellows of flexible material mounted to the U-shaped, rigid frame to partially enclose the bounded space. Teeth 213 can extend from the U-shaped, rigid frame away from the partially enclosed space. Jaw 212B can be configured similarly with teeth 213 configured to mesh with teeth 213 of jaw 212A. Thus, the flexible material of jaws 212A and 212B can form a full enclose when jaws 212A and 212B are rotate to engage, but can bend to not interfere with teeth 213 engaging target tissue 254.

Teeth 213 can be configured to have one or more orientations. For example, teeth 213 can be angled distally toward target tissue 254, or proximally toward shaft 222. In examples, some of teeth 213 can be angled proximally and some of teeth 213 can be angled distally. In examples, teeth 212 can be oriented in different directions.

As discussed above, components or portions of tissue collection device 204 can be made of optically enhanced materials. In examples, jaws 212A and 212B can be made of translucent or transparent material that can allow light waves to travel therethrough, thereby allowing lens 238 to “see through” jaws 212A and 212B. Transparent materials can allow lens 238 to see native coloring of target tissue 254. Translucent materials can be configured to allow lens 238 to see target tissue 254 in a filtered manner. As such, jaws 212A and 21B can be translucently tinted with different colors to enhance viewing of certain tissue types or mute viewing of other tissue types.

However, in order to maintain control of tissue collection device 204, e.g., to maintain accurate employment of teeth 213, portions of tissue collection device 204 can be opaque, reflective or translucent. In particular, teeth 213 can be made of opaque, reflective or translucent material or can have a coating applied thereto. In examples, teeth 213 can be opaque to be easily viewable by lens 238. In additional examples, teeth 213 can be configured to optically interact with light from lens 239. For example, teeth 213 can have a reflective coating applied thereto, such a coating of grains of reflective particles or titanium oxide. Thus, light from lens 239 can be bounced bac to lens 238. In additional examples, teeth 213 can be fluorescent to light up when engaged by a certain type of light. Thus, light from lens 239 can cause lens 238 to view teeth 213 in a particular wavelength that is more discernable relative to duct 255. In examples, only some of teeth 213 can be reflective or fluorescent.

In view of the foregoing, use of optically enhanced tissue collection devices can facilitate viewing of target tissue 254 through jaws 212A and 212B, viewing of sample tissue 258 within jaws 212A and 212B, and viewing of laceration 260 where sample tissue 258 was removed from target tissue 254. As such, endoscope 230 can be used to view interior tissue layers within laceration 260 and potentially diagnose conditions of the that tissue.

FIG. 7A is a schematic illustration of tissue retrieval device 300 comprising boring device 302, which can be inserted into endoscope 304. FIG. 7B is side view of tissue retrieval device 300 of FIG. 7A with boring device 302 shown in cross-section to show storage space 306 with sample tissue 308. FIG. 7A and 7B are discussed concurrently and the components therein are not necessarily drawn to scale.

Tissue retrieval device 300 can further comprise shaft 310. Boring device 302 can comprise container 312, boring lands 314, blade 316 and bore 318. Endoscope 304 can be configured similarly as endoscope 230 of FIGS. 6A and 6B and can comprise another example of auxiliary scope 134. Endoscope 304 can comprise shaft 320, working channel 322, passage 324 and lens 326. Field of view 328 can project from lens 326. Endoscope 304 can further comprise light lens 329 for projecting light of one or more wavelengths onto target tissue 330.

Tissue retrieval device 300 can be configured to engage target tissue 330 in the axial direction of arrow B. For example, tissue retrieval device 300 can be positioned in front of a mound or protrusion of tissue or proximate a wall of tissue. Shaft 306 can be advanced in the direction of arrow B by a user to engage target tissue 330. Boring device 302 can be configured as a punch. Container 312 can have a cone shape and can include distal bore 318 that can be configured to push through tissue. Thus, tissue retrieval device can be configured to punch through tissue to take a tissue sample similar to core sampling a tree, etc. The distal or leading edge of bore 318 can be sharpened. In such a configuration, lands 314 and blade 316 can be omitted from container 312.

In examples, boring device 302 can be configured as an auger. As such, container 312 can have a cone shape with lands 314 wrapped around container 312 in a spiral manner. Lands 314 can be configured to engage tissue to allow container 312 to penetrate the tissue in the direction of arrow B. In some situations, it is possible for boring device 302 to slip over the target tissue, such as due to slippery or moist conditions. Thus, it can be difficult or impossible to engage the tissue sufficiently to collect a desirable volume of sample tissue. Lands 314 can be configured to facilitate engagement with the tissue. Shaft 306 can be rotated by an operator to rotate container 312 and lands 314. Lands 314 can grab tissue while being rotated to cause further axial penetration of boring device 302 into the tissue. As such, as boring device 302 is advanced forward, the distal tip of container 312 can maintain engagement with the tissue. As container 312 enters tissue, blade 316 can be configured to slice or shave tissue away from the patient. Blade 316 can comprise a sharpened edge of an opening in container 312 and can be configured similar to a potato peeler. In examples, only one of blade 316 and bore 318 can be used. However, both can be included as illustrated.

Additionally, In the various examples, container 312 can be configured to have an internal space to capture and retain sample tissue collected by bore 318 and/or blade 316.

As discussed herein, features of boring device 302 can be optically enhanced to interact with point of view of lens 326 and light being emitted at light lens 329. For example, container 312 can be fabricated from transparent or translucent material. As such, line of sight 340 can extend from lens 326 through container 312 to laceration 342 where sample tissue 308 was removed from target tissue 330. Additionally, line of sight 344 can extend from lens 326 through container 312 to sample tissue 308 within container 312.

Other features of boring device 302 can be configured to interact with light from lens 329. For example, boring lands 314, blade 316 and bore 318 can be fabricated from or coated with material to reflect light or to be luminescent.

Thus, as discussed herein boring device 302 can be optically enhanced to hide or make invisible portions of the device by being transparent or translucent and to visually brighten or highlight other portions of the device by being reflective or luminescent. Thus, portions of boring device 302, such as those not functionally important to identifying and removing target tissue, can be optically minimized to reduce noise in imaging signals for an operator, and portions of boring device 302, such as those that are functionally important to identifying and removing target tissue, can be optically maximized to increase visibility in imaging signals for an operator.

FIG. 8A is a schematic illustration of endoscopy system 400 comprising endoscope 402 and biopsy instrument 404. Biopsy instrument 404 can be tethered to the distal end portion of endoscope 402 for insertion into anatomy of a patient, thereby facilitating collection of large volumes of sample tissue without having to reinsert endoscope 402 into the patient multiple times.

Biopsy instrument 404 can comprise a device configured for the separation, collection and/or retrieval of biological matter, such as tissue, from a patient. In an example, biopsy instrument 404 can be configured as forceps shown in FIG. 8B. Biopsy instrument 404 can comprise separator 406, which, in the illustrated example, comprises jaws 408A and 408B, hinge 410, base 412, control cables 414A and 414B and couplers 416A and 416B. Biopsy instrument 404 can additionally comprise handpiece 418 and couplers 420A and 420B. Handpiece 418 can be operatively coupled to control unit 16 (FIGS. 1 and 2) via connector 421 and cable 419.

Endoscope 402 can comprise shaft 422, lumen 424, handpiece 426, control 428, connector 430 and cable 432. Handpiece 426 can comprise a controller for operating the functions of endoscope 402. For example, control 428 can comprise a knob for activating pull wires within shaft 422. Handpiece 426 can be connected to system control unit 16 (FIGS. 1 and 2) via cable 432 and the use of connector 430. The components illustrated in FIG. 8A are not necessarily drawn to scale.

Endoscope 402 can include components and features as are described with reference to endoscope 230 and endoscope 304 of FIGS. 5B-7B. Endoscope 402 can include steering capabilities (e.g., pull wires), illumination capabilities (e.g., a light emitter), guidance capabilities (e.g., a camera or imaging system) and fluid capabilities (e.g., irrigation and suction capabilities). In particular, endoscope 402 can be configured to operate with a working tool using lumen 424. Lumen 424 provides connection between distal-most end 434 of shaft 422 and handpiece 426 such that an instrument can be inserted into lumen 424 to function within anatomy through the distal end of shaft 422 and to be controlled at proximal end 436 of endoscope 402 via handpiece 426.

Biopsy instrument 404 can comprise a working tool configured to retrieve, remove and collect biological matter from within a patient. In the illustrated example, biopsy instrument 404 comprises forceps. However, other biopsy instruments or working tools can be used, such as boring device 302 of FIGS. 7A and 7B, as well as the other devices described herein.

Base 412 can comprise a component upon which to mount separator 406 and that can engage shaft 422. In examples, base 412 can be configured to abut distal-most end 434 to be held in place by control cables 414A and 414B. In other examples, base 412 can be configured to be coupled to distal-most end 434, such as via a threaded coupling, a protrusion that can be interference fit with lumen 424, a quick connect coupling or a magnetic coupling. Hinge 410 can comprise an axle or pivot point mounted to base 412 upon which one or both of jaws 408A and 408B can pivot. Jaws 408A and 408B can thus be mounted to hinge 410. Control cables 414A and 414B can extend from jaws 408 and 408B through, alongside or around base 412 for extension into lumen 424. Control cables 414A and 414B can comprise various devices or components allowing for remote, e.g., proximal, control of biopsy instrument 404. In examples, control cables 414A and 414B can comprise wires or cables configured to pull on components of biopsy instrument 404. In the illustrated example, two control cables are shown for manipulation of jaws 408A and 408B. However, only one control cable can be used or more than two control cables can be used.

Proximal ends of control cables 414A and 414B can be provided with couplers 416A and 416, respectively. Couplers 416A and 416B can be connected with couplers 420A and 420B of handpiece 426. The union of couplers 416A and 416B with couplers 420A and 420B, respectively, can allow the transmission of actuation force through control cables 414A and 414B to biopsy instrument 404 from handpiece 426. Thus, handpiece 426 can be operated or can include button, knobs, levers and the like, to pull and push control cables 414A and 414B. In examples, couplers 416A and 416B can comprise plugs and couplers 420A and 420B can comprise sockets. In examples, couplers 416A and 416B can comprise loops or eyelets and couplers 420A and 420B can comprise latches, clips, hooks and the like, or vice versa.

Biopsy instrument 404 is shown in FIG. 8A as a mechanically actuated biopsy instrument. However, in examples, an electrically actuated biopsy instrument can be provided in which one or more control cables are configured to deliver at least one of power and control signals to the biopsy instrument. As such, the biopsy instrument can comprise an electrically activated device, e.g., via an electric motor or actuator. Correspondingly, handpiece 426 can comprise appropriate actuators for operating electrical components of such a biopsy device, such as buttons, switches and the like.

Typically, an endoscope is inserted into the anatomy of a patient and then the working tool is inserted through the endoscope. As such, as discussed above, the working tool, and particularly the distal, functioning end of the working tool, must be sized to fit within the lumen of the endoscope, which limits the size of the functional end and the working tool disposed thereat, as the working lumen is necessarily smaller than the cross-section of the endoscope. As mentioned above, a typical working tool lumen such as lumen 424 can be configured to have a diameter of approximately 1.2 mm.

With the devices and systems of the present disclosure, a working tool can comprise a functional element that is larger than a typical working tool lumen of an endoscope by providing a working tool that can be attached pre-insertion to the distal end of the endoscope. The working tool lumen can be used for the passage of control elements from the working tool that can be coupled proximally to a controller or handpiece for the working tool. The working tool can be sized larger than the working tool lumen and can extend radially, relative to the longitudinal axis of the endoscope, beyond the working tool lumen. To facilitate such capabilities, the working tool can include components that are fabricated of materials that allow for the passage of light (e.g., transparent or translucent materials) in order to minimize obstruction of imaging and illuminating capabilities of the endoscope.

Biopsy instrument 404 can be coupled to endoscope 402 via insertion of couplers 416A and 416B into lumen 424 at distal-most end 434. Couplers 416A and 416B can be extended through shaft 422 and handpiece 426 to extend from proximal end 436. Base 412 can be abutted to shaft 422 and, in examples, mounted thereto. Couplers 416A and 416B can be linked with couplers 420A and 420B of handpiece 418. Handpiece 418 can be mounted to handpiece 426 via any suitable coupling, such as threaded fasteners, snap fit couplers, hook and loop fastener material and the like. In an example, tension applied to control cables 414A and 414B between base 412 and handpiece 418 by the joining of couplers 416A and 416B and couplers 420A and 420B, can be sufficient to join biopsy instrument 404 and handpiece 418 to endoscope 402. Configured as such, separator 406 can be tethered to shaft 422. However, separator 406 can be attached with other tethering arrangements, such as those discussed herein with reference to base 412.

Once assembled, biopsy device 404 can be positioned at distal-most end 434 to be manipulated at a proximal end by a user. Jaws 408A and 408B can be sized larger than lumen 424, thereby having larger internal volumes that permit larger volumes of tissue samples to be acquired. In order to facilitate operation of biopsy device 406 that is larger than lumen 424, which can potentially obstruct lenses 238 and 239 (FIG. 6A), jaws 408A and 408B can be made of light transmitting material, as is described throughout the present application. Furthermore, in order to facilitate collection of a large volume of sample biological matter, e.g., via executing multiple collection operations (e.g., “bites”) with forceps comprising biopsy instrument 404.

FIG. 8B is a side view of forceps 438 suitable for use as a biopsy device of the present disclosure. Forceps 438 can comprise base 440, jaws 442A and 442B, hinge 444, actuators 446A and 446B and control wires 448A and 448B. Forceps 438 is described with reference to engagement with endoscope 230 of FIG. 5B for the sake of illustration of imaging lens 238 and illumination lens 239. Base 440 can be configured to engage shaft 232. Base 440 can abut the distal-most face of shaft 232 and can be configured to be taller than height H1 of working channel 234, thereby preventing base 440 being capable of entering working channel 234. In examples, base 440 can mate flush with shaft 232 to provide a stable connection to shaft 232, thereby inhibiting rocking or vibration, and allowing jaws 442A and 442B to firmly engage target tissue. As mentioned, base 440 can additionally be configured to be positively held in place relative to shaft 232 via a mechanical coupling or the like.

Hinge 444 can comprise a connection point for jaws 442A and 442B to couple to base 440. Hinge 444 can comprise a round pin or shaft over which corresponding bores in jaws 442A and 442B can be fit. Thus, jaws 442A and 442B can be configured to freely rotate on hinge 444. However, rotation of jaws 442A and 442B on hinge 444 can be controlled by control wires 448A and 448B. Control wires 448A and 448B can be coupled to actuators 446A and 446B, respectively, of jaws 442A and 442B. Actuators 446A and 446B can comprise levers extending at angle from jaws 442A and 442B relative to a centerline of working channel 234. Thus, control wires 448A and 448B can be operated by handpiece 418 to pull actuators 446A and 446B to rotate jaws 442A and 442B about hinge 444 to facilitate collection of tissue samples. In examples, control wires 448A and 448B can be pre-curved to impart rotational bias to actuators 446A and 446B to an open or closed position. However, in examples, actuators 446A and 446B can be provided with other biasing elements, such as springs. As such, pulling of control wires 448A and 448B can cause closing or opening of jaws 442A and 442B, as desired. As illustrated, jaws 442A and 442B can include teeth to facilitate cutting and tearing of tissue away from the anatomy. Though the illustrated example is shown with reference to actuators comprising levers, other actuators, such as pull rods or screw mechanisms, can be used.

As illustrated, jaws 442A and 442B can extend radially beyond height H1 of working channel 234 so as to obstruct lenses 238 and 239. In an example, working channel 234 can have height H1 of 1.2 mm. In particular, jaw 442A can extend radially above working channel 234 to be positioned between lenses 238 and 239 and target tissue distal of endoscope 230. As such, in order to prevent jaws 442A and 442B from preventing lenses 238 and 239 from providing guidance and target tissue acquisition to endoscope 230, such as by providing imaging of tissue, jaws 442A and 442B can be made of material that allows light to pass therethrough, such as transparent, translucent and semi-opaque material, as is described herein. As such, jaws 442A and 442B can be larger than working channel 234 without interfering with operation of endoscope 230.

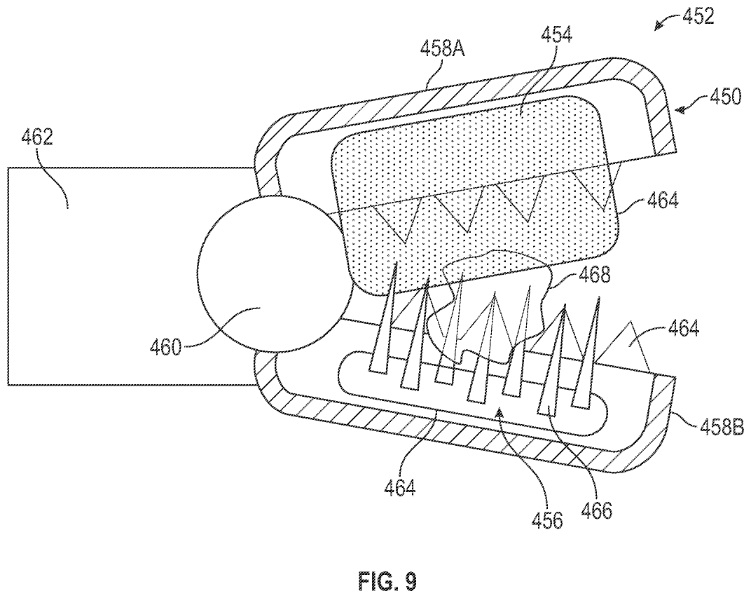

FIGS. 9-11 illustrate examples of a biopsy instrument suitable for use with the present disclosure. FIGS. 9-11 illustrate simplified schematic views of forceps 438 of FIG. 8B. However, other tissue collection or retrieval devices and other forceps configurations can be used.

FIG. 9 is a schematic illustration of biopsy instrument 450 comprising forceps 452 having a tissue retention system comprising sponge 454 and needle array 456. Forceps 452 can comprise jaws 458A and 458B, hinge 460 and base 462. Jaws 458A and 458B can include teeth 464. Needle array 456 can comprise base 464 and needles 466. Tissue sample 468 can be located between jaws 458A and 458B. Sponge 454 can comprise a resiliently deformable body that can be deformed by the presence of sample tissue within jaws 458A and 458B, but that tends to retain its shape to apply a retaining force to the sample tissue. Needles 466 can comprise tines or pins configured to pierce sample tissue and sponge 454.

Sponge 454 and needle array 456 can comprise a capacity enhancement feature that allows jaws 458A and 458B to hold a larger volume of sample tissue than without sponge 454 and needle array 456. Sponge 454 can be attached to the internal cavity of jaw 458A, such as via adhesive or any suitable manner, and used to bias tissue sample 468 toward 458B. Base 464 can be attached to the internal cavity of jaw 458B, such as via adhesive or any suitable manner. As such, jaws 458A and 458B can be used to obtain tissue sample 468 and position tissue sample 469 between jaws 458A and 458B, such as by using control wires 448A and 448B. Thereafter, jaws 458A and 458B can be reopened to obtain an additional tissue sample, and sponge 454 can push tissue sample 468 into needles 466 to prevent tissue sample 468 from falling out of forceps 452. In examples, sponge 454 and needle array 456 can be used independently (e.g., one without the other) to retain tissue sample 468 between jaws 458A and 458B.

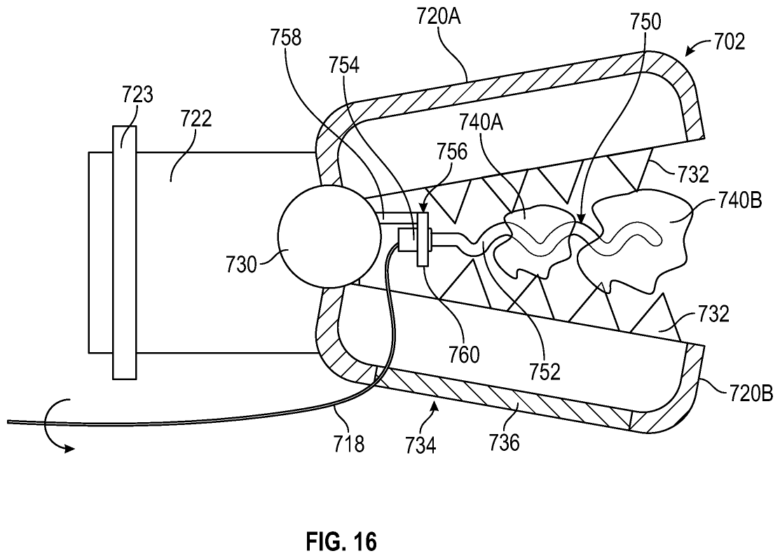

As mentioned below, needle array 456, including base 464 and needles 466, can be configured as or incorporated into a tissue retrieval shuttle. For example, base 464 can be attached to tether 718 and base 464 can be dislodged from jaw 720B. For example, base 464 can be attached to jaw 720B with an adhesive that can be broken when tether 718 is pulled with sufficient force. Base 464 and needles 466 can be pulled through a portal or door within jaw 720B, such as door 736 of FIG. 16, or pulled out from between jaws 720A and 720B. In examples, needle array 456 can be provided with additional features to facilitate being operating as aa shuttle. For example, needle array 456 can be provided with fencing or barriers to prevent the tips of needles 466 from undesirably engaging tissue while being pulled proximally from anatomy. The fencing can extend beyond the tips of needles 466, such as by extending from base 464 a greater length. In examples, needles 466 can be curved to prevent sliding along tissue. For example, needles 466 can be curved distally such that the tips of needle 466 generally point in the distal direction. As such, tissue separated by jaws 720A and 720B can move proximally onto the tips of needles 466, and needles 466 can slide proximally along tissue and other objects. In additional examples, needles 466 can be flexible to inhibit pricking of anatomic tissue while being retrieved, but can have sufficient rigidity to allow tissue collected by jaws 720A and 720B to be pushed onto needles 466.

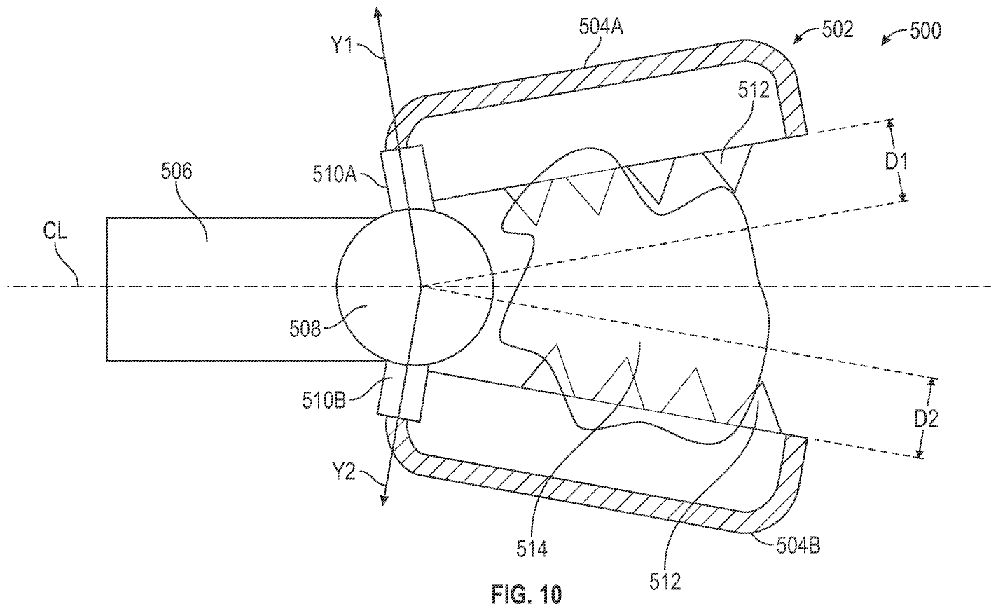

FIG. 10 is schematic illustration of biopsy instrument 500 comprising forceps 502 having expandable jaws 504A and 504B. Forceps 502 can comprise base 506, hinge 508 and rails 510A and 510B. Jaws 504A and 504B can include teeth 512. Tissue sample 514 can be located between jaws 458A and 458B. Jaw 504A can be slidably coupled to rail 510A so as to be displaceable in direction Y1. Thus, jaw 504A can be displaced distance DI from centerline CL (relative to not being rotated). Similarly, Jaw 504B can be slidably coupled to rail 510B so as to be displaceable in direction Y2. Thus, jaw 504B can be displaced distance D2 from centerline CL (relative to not being rotated).

Jaws 504A and 504B can be used to obtain tissue sample 514, such as via actuation by control wires 448A and 448B. Jaws 504A and 504B can be moved radially outward in the direction of arrows Y1 and Y2. In an example, jaws 504A and 504B can be moved on rails 510A and 510B by resistance from tissue sample 514. Jaws 504A and 504B can include tracks that ride in rails 510A and 510B. Thus, upon the presence of tissue sample 514 when jaws 504A and 504B are being actuated to be closed, jaws 504A and 504B can move outwardly to accommodate the presence of tissue sample 514. The tracks can ride in rails 510A and 510B with an appropriate level of friction to prevent free movement therebetween. Jaws 504A and 504B can thus be moved to accommodate the collection of multiple tissue samples or larger sized samples as compared to jaws that are fixed at the pivot point.

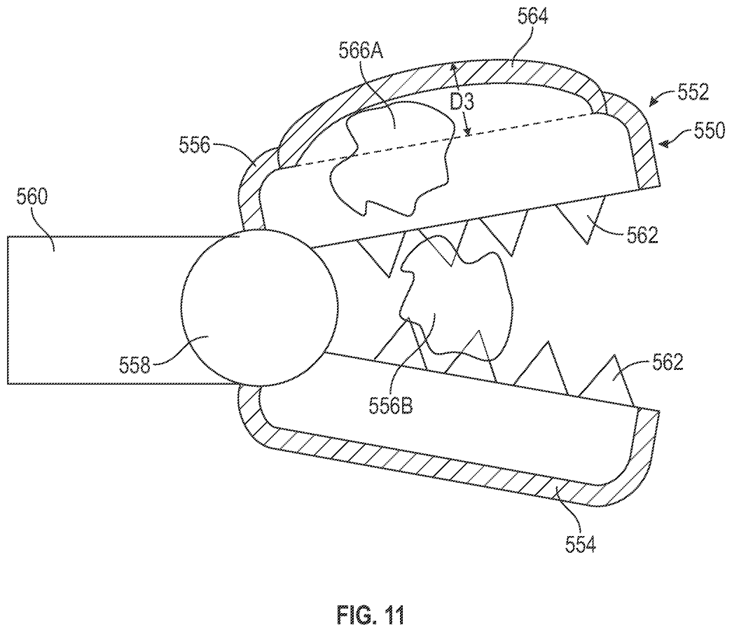

FIG. 11 is a schematic illustration of biopsy instrument 550 comprising forceps 552 having flexible jaw 554 and opposing jaw 556. Flexible jaw 554 and opposing jaw 556 can be connected at hinge 558 and coupled to base 560. Jaws 554 and 556 can include teeth 562. Flexible jaw 554 can comprise deflectable wall 564. Tissue samples 566A and 566B can be located between jaws 554 and 556.

Jaws 554 and 556 can be used to obtain tissue sample 556A, such as via actuation by control wires 448A and 448B. Thus, tissue sample 556A can be positioned between jaws 554 and 556. Tissue sample 556A can occupy the space between jaws 554A and 554B. However, rather than stopping the tissue collection procedure to withdraw biopsy instrument 550 and the endoscope in which it is inserted, jaws 554 and 556 can be operated to collect second tissue sample 556B, which can be positioned between jaws 554A and 556. The presence of tissue sample 556B can displace tissue sample 556A outward toward jaw 556. Tissue sample 566A can deflect deflectable wall 564 outward away from hinge 558, distance D3 from an undeflected position, to produce more space between jaws 554 and 556.

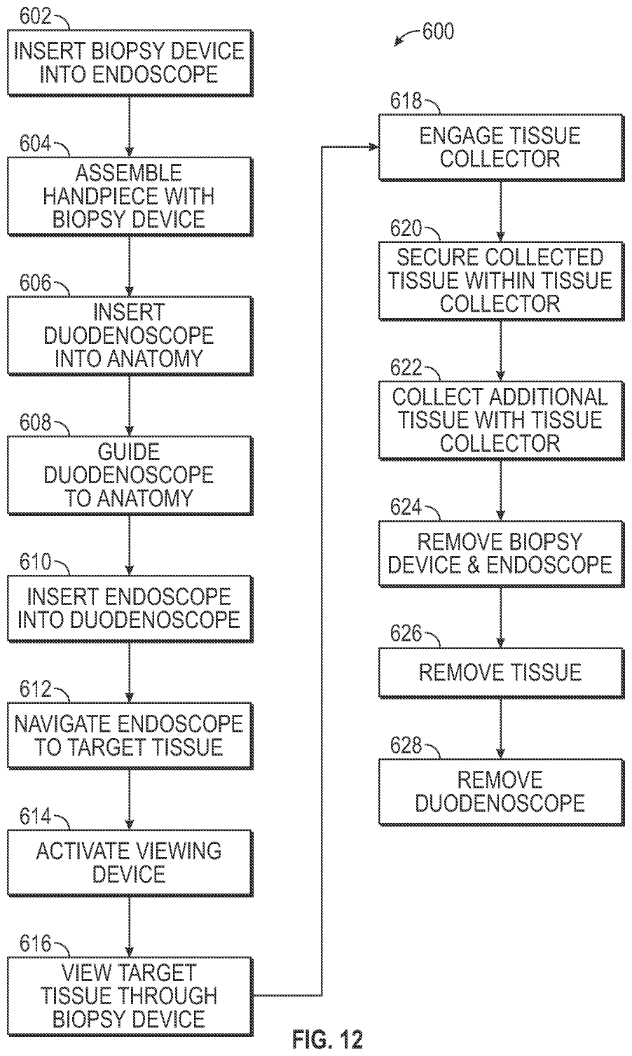

FIG. 12 is a block diagram illustrating examples of method 600 of collecting biological matter from a patient using the biopsy devices and tissue retrieval devices of the present disclosure, such as those that are tethered distally of an endoscope. Method 600 can encompass the use of endoscopy system 400 of FIG. 8A, forceps 438 of FIG. 8B, biopsy instrument 450 of FIG. 9, biopsy instrument 500 of FIG. 10 and biopsy instrument 550 of FIG. 11, as well as other instruments including those described herein.

At step 602, a biopsy device, such as forceps 438 of FIG. 8B, biopsy instrument 450 of FIG. 9, biopsy instrument 500 of FIG. 10 and biopsy instrument 550 of FIG. 11, can be inserted into endoscope 402. For example, control cables 414A and 414B can be inserted into lumen 424 of shaft 422. In examples, control cables 414A and 414B can be longer than endoscope 402 such that proximal ends of control cables 414A and 414B having couplers 416A and 416B can extend proximally out of endoscope 402.

At step 604, the biopsy device can be attached to the endoscope to prevent separation therefrom. For example, handpiece 418 can be assembled to handpiece 426 to prevent control cables 414A and 414B from sliding out of lumen 424, such as by attaching couplers 420A and 420B to couplers 416A and 416B. In other examples, couplers 416A and 416B can be attached to handpiece 426 without the use of handpiece 418. Additionally, base 412 of biopsy device 406 can be attached to shaft 422 of endoscope 402.

At step 606, the duodenoscope can be inserted into anatomy of a patient, such as by being inserted into an opening or incision in the patient. In examples, the duodenoscope can be guided to a duodenum of the patient to perform a cholangioscopic procedure. However, the tethered biopsy devices of the present disclosure can be used in other types of procedures referenced herein, such as other gastrointestinal procedures and renal area procedures.

At step 608, the duodenoscope can be inserted into and navigated through anatomy of a patient. For example, endoscope 14 (FIG. 1) can utilize native imaging capabilities to guide insertion section 28 through anatomic ducts of the patient. Insertion section 28 can be bent or curved using control knob 38 to facilitate turning of endoscope 14.

At step 610, an endoscope or auxiliary scope can be inserted into the duodenoscope to access anatomy located further in the duct. For example, endoscope scope 402 (FIG. 8A), with biopsy device 404 attached thereto, can be inserted into lumen 62 (FIG. 3C) or lumen 132 (FIG. 4) to reach another anatomic duct intersecting the anatomic duct reached by endoscope 14. Elevator 54 (FIG. 3C) can be used to bend or turn endoscope 402.

At step 612, the endoscope can be navigated through the anatomy. For example, endoscope 402 can be guided from the duodenum to the common bile duct. The endoscope can be guided using native steering and imaging capabilities of the endoscope.

At step 614, a viewing device or imaging device on the auxiliary scope can be activated in order to view biological matter of the patient. For example, imaging unit 110 can be activated to view anatomy in field of view 240 of lens 238. Images can be sent back to control unit 16.

At step 616, target tissue can be viewed using an imaging unit and a video display monitor. For example, imaging unit 110 can use lens 238 to display target tissue on output unit 18. Lens 238 can view the target tissue through transparent or translucent portions of the tissue collection devices, such as forceps 438 of FIG. 8B, biopsy instrument 450 of FIG. 9, biopsy instrument 500 of FIG. 10 and biopsy instrument 550 of FIG. 11. Light from a light source can be used to illuminate the target tissue. For example, light from lens 239, as generated by lighting unit 112, can be directed upon the target tissue. As discussed herein, various components of the tissue collection devices can be configured to reflect light from the light source to enhance visibility.

At step 618, a tissue collector of the biopsy device can be navigated to the location of target tissue within the patient. For example, jaws 408A and 408B can be navigated through an anatomic duct to target tissue 254 (FIG. 6A). The target tissue can comprise tissue that is potentially diseased or otherwise indicative of a diseased condition of the patient. Jaws 408A and 408B can be pushed, pressed or otherwise brought into pressurized contact with the target tissue. Thus, jaws 408A and 408B can be rotated about hinge 410 by activation of guide cables 414A and 414B from handpiece 418 to cause jaws 408A and 408B to slice, punch, shave, etc. one or more pieces of tissue away from the anatomy of the patient. Furthermore, portions of the tissue collection device can interact with light from lens 239 to enhance visibility of such portions. For example, tissue separating components, such as teeth or blade edges, can be reflective or luminescent to enhance display on the video display monitor, such as output unit 18.

At step 620, sample tissue or biological matter separated or collected from the patient at step 618 can be stored within a space or internal volume inside the tissue collection device. For example, separated sample tissue 258 can be positioned within space 256. As explained with reference to biopsy instrument 450 of FIG. 9, biopsy instrument 500 of FIG. 10 and biopsy instrument 550 of FIG. 11, the tissue retrieval devices can include capacity enhancing features that facilitate the collection of large volumes of sample tissue, such as multiple sample tissue volumes. Sponge 454 and needle array 456 can comprise retention features that can operate independently or cooperatively to bias or retain collected tissue samples within jaws 458A and 458B to prevent dislodging of the collected tissue samples. Slidable rails 510A and 510B, and deflectable wall 564 can comprise capacity increasing features that can be employed to secure increasingly larger volumes of tissue within their respective jaws.

At step 622, additional tissue can be collected with the biopsy device by reapplying the tissue separator device. As more tissue pieces are collected, the newly collected pieces can push the previously collected pieces further into the tissue retrieval device. The previously collected pieces can then activate the capacity enhancing features, such as by the previously collected pieces being pushed into engagement with sponge 454, being pushed into needle array 456, pushing movable jaws 504A and 504B outward, and moving deflectable wall 564.

At step 624, the biopsy device can be removed from the patient, such as by removal from the duodenoscope, which can be left in place inside the anatomy. Safeguards can be put into place to ensure removal of the tissue collection device without inadvertently cutting anatomy of the patient.

At step 626, the collected sample tissue can be removed from the tissue collection device. For example, jaws 408A and 408B can be rotated away from each other to access space therebetween and remove sample tissue for analysis, etc.

At step 628, the duodenoscope can be removed from the patient. The patient can thereafter be appropriately closed up or prepared for completion of the procedure.

As such, method 600 illustrates examples of a method of collecting biological matter from internal passages of a patient in large enough quantities, e.g., by using an optically enhanced (e.g., transparent, clear, reflective, translucent, luminescent, or scattering) tethered tissue removal device with internal storage, to eliminate or reduce insertion and removal of surgical devices from the patient. Tethering of the tissue removal device allows for a larger instrument to be used than the working channel or lumen of an endoscope can allow. The optical enhancements allow the tissue removal device to be at least partially invisible to a camera and recognized by a light source, for example.

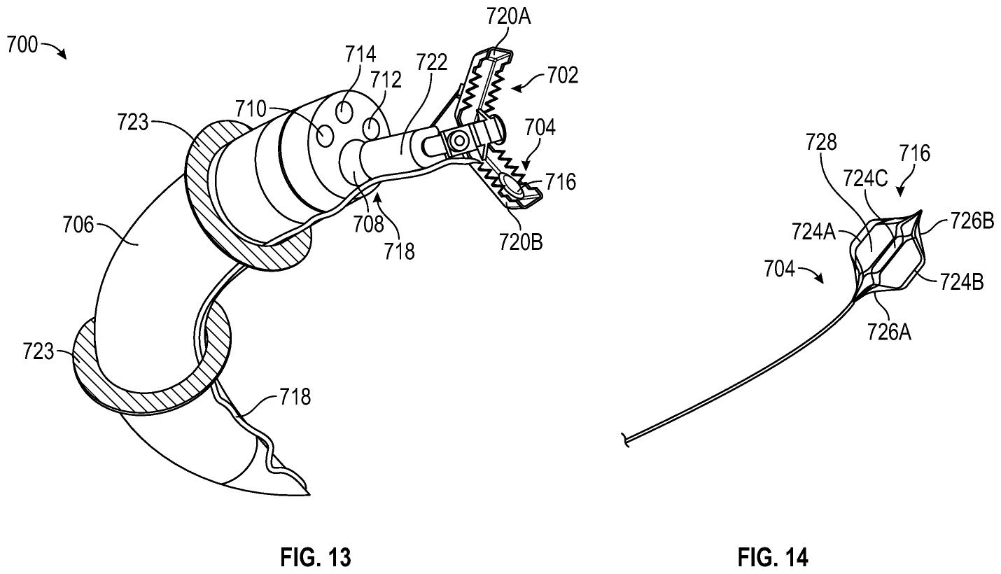

FIG. 13 is perspective view of a scope 700 having tissue collector 702 and tissue shuttle 704. Scope 700 can comprise shaft 706, working lumen 708, as well as one or more treatment, guidance or navigate features, such as light emitter 710, imaging device 712 and irrigation channel 714. Tissue shuttle 704 can comprise cage 716 and retriever 718. Shaft 706 can comprise one or more fasteners 725 for securing retriever 718. Tissue collector 702 can comprise jaws 720A and 720B and base 722. As described below, tissue shuttle 704 can comprise a device held within jaws 720A and 720B to hold tissue samples collected by tissue collector 702. Retriever 719 can be activated, e.g., pulled, from a proximal portion of scope 700 to withdraw tissue shuttle 704 from jaws 720A and 720B to obtain the collected sample tissue and permit jaws 720A and 720B to obtain an additional tissue sample.

Scope 700 can be configured similar to any of the devices described herein, such as auxiliary scope 134 of FIG. 4, auxiliary endoscope 230 of FIG. 5B and endoscope 402 of FIG. 8A, with the addition of fasteners 725. Tissue collector 702 can be configured similar to any of the devices described herein, such as tissue collection device 204 of FIG. 5B, biopsy instrument 404 of FIG. 8A, biopsy instrument 500 of FIGS. 10 and 550 of FIG. 11. In a particular example, tissue collector 702 can comprise an embodiment of biopsy instrument 404 where tissue shuttle 704 can be pre-loaded into jaws 720A and 720B and tissue collector 702 can be pre-assembled with endoscope 402 (FIG. 8A) before insertion into anatomy.

FIG. 14 is a perspective view of tissue shuttle 704 of FIG. 13A. Tissue shuttle 704 can comprise a receptacle for receiving and storing tissue samples collected by jaws 720A and 720B. Thus, the receptacle can include various ways to allow the collected tissue samples to enter therein. In the example of FIG. 14, the receptacle of tissue shuttle 704 can comprise cage 723, including wires 724A, 724B and 724C connected at hinges 726A and 726B. Space between wires 724A-724C, plus other wires not seen in FIG. 14, can allow tissue collected by jaws 720A and 720B to pass into space 728 within wires 724A-724C. Wires 724A-724C can be flexible to allow jaws 720A and 720B to collapse cage 723 when jaws 720A and 720B are fully closed, such as to facilitate insertion of tissue collector 702 into anatomy, and to allow jaws 720A and 720B to push tissue samples passed wires 724A-724C into space 728. However, an adequate number of wires can be provided such that when expanded with tissue samples, the wires allow the tissue sample to be retained therein.

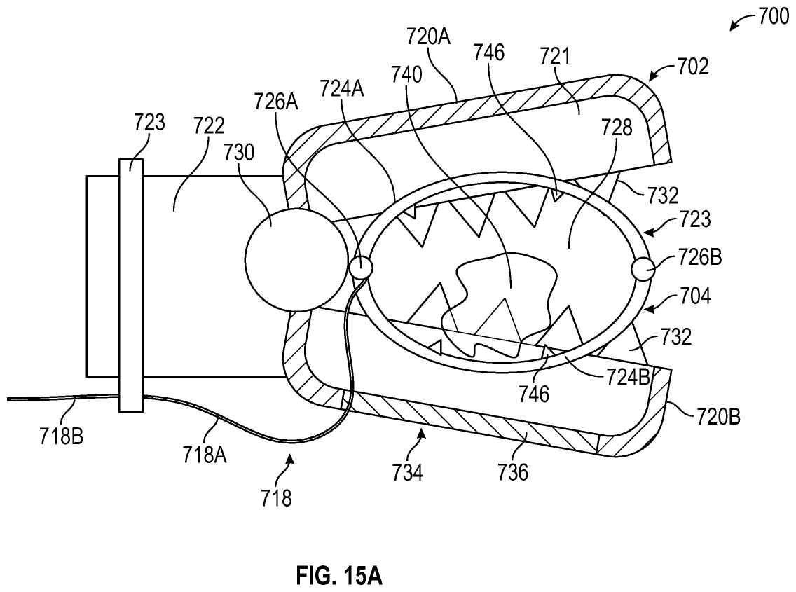

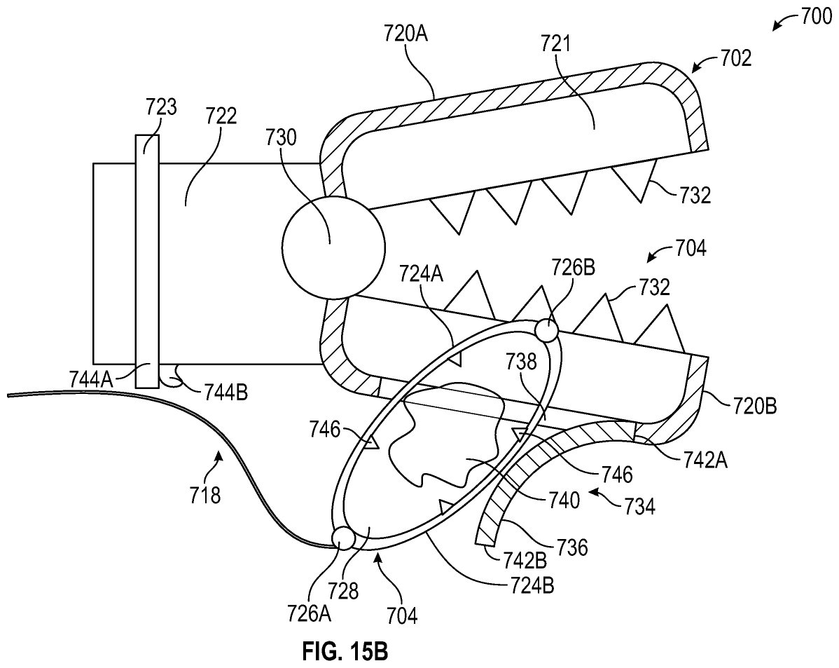

FIG. 15A is a schematic side view of tissue collector 702 of FIG. 13 with tissue shuttle 704 disposed inside of jaws 720A and 720B. Tissue collector 702 can comprise a forceps having base 722 and jaws 720A and 720B connected at hinge 730. Jaws 720A and 720B can form space 721 therebetween and can include teeth 732. Tissue collector 702 can further comprise portal 734, which, in the illustrated example, can comprise door 736 on jaw 720B. Tissue shuttle 704 can comprise wires 724A and 724B connected by hinges 726A and 726B. Tissue sample 740 can be located between jaws 720A and 720B.

Base 722 can comprise a portion of shaft 706 used to manipulate jaws 720A and 720B. Base 722 can additionally be similar to base 412 of FIG. 8A. Hinge 730 can connect jaws 720A and 720B to base 722 in a pivotable fashion. In either configuration, controls at a proximal end of shaft 706 can be used to actuate jaws 720A and 720B to collect tissue samples. Teeth 732 can be used to slice into tissue to separate tissue samples from anatomy and deposit the tissue samples in space 721 between jaws 720A and 720B. Tissue collector 702 can therefore comprise a tissue separator.

Tissue shuttle 704 can be positioned in space 721. In the illustrated example, cage 723 can be floating between jaws 720A and 720B. In additional examples, cage 723 can be attached to one or both of jaws 720A and 720B. In particular, cage 723 can be releasably attached to one or both of jaws 720A and 720B such that pulling of retriever 718 can cause cage 723 to detach from one or both of jaws 720A and 720B. In examples, cage 723 can be attached to jaws 720A and 720B with an adhesive. In examples, cage 723 can be attached to jaws 720A and 720B with frangible straps, hook and loop fastener material and the like. With wires 724A and 724B attached to jaws 720A and 720B, pivoting of jaws 720A and 720B at hinge 730 can cause wires 724A and 724B to expand and contract at hinges 726A and 726B. Thus, jaws 720A and 720B can be configured such that, as jaws 720A and 720B rotate apart wires 724A and 724B can move apart to increase the space between adjacent wires to allow a tissue sample to enter space 728. When jaws 720A and 720B move closer together, wires 724A and 724B can be moved closer together, e.g., dispersed at more even intervals, spread apart to decrease the space between adjacent wires. In additional examples, wires 724A and 724B can be telescopic in nature to allow expansion and contraction. In examples, a gearing mechanism can be used to convert rotation at hinge 730 to provide contraction and expansion forces to wires 724A and 724B. In examples, cage 723 can include four wires wherein two neighboring wires can be configured to lay against each other such that two touching wires are spaced one-hundred-eighty degrees From two other touching wires, but can be spring loaded to space four wires ninety degrees From each other.

One or both of jaws 720A and 720B can be provided with an access point or door to allow entry and egress into space 721 with jaws 720A and 720B rotated to a closed position. Portal 734 can comprise door 736. Door 736 can be located in aperture 738 (FIG. 15B) of jaw 720B. Aperture 738 can comprise a cut-out or aperture in jaw 720B. Door 736 can close off aperture 738 to prevent shuttle 704 from passing through aperture 738, as discussed with reference to FIG. 15B. In additional examples, door 736 can be flexible or can include a flexible portion surrounded by a frame to allow for expansion of the space between jaws 720A and 720B to facilitate collection of additional tissue, similar to what is shown in FIG. 11.

Retriever 718 can extend from cage 723 out of space 721 toward base 722. Retriever 718 can comprise a component or device configured to extract shuttle 704 from space 721. For example, retriever 718 can be pulled to dislodge shuttle 704 from between jaws 720A and 720B. In examples, retriever 718 can comprise a flexible tether, such as a rope or cord made from a plastic, polymer or another biocompatible material. In examples, retriever 718 can comprise a stiff yet flexible rod. In examples, retriever 718 can comprise a rigid rod that is shaped to conform to a desired pre-curve or shape. In examples, retriever 718 can a two-piece device having flexible distal portion 718A and stiff or rigid proximal portion 71B.

Proximal portion 718B can be affixed to base 722 and shaft 706 using fasteners 725. A plurality of fasteners 725 can be spaced along shaft 706 at regular or irregular intervals. Fasteners 725 can comprise means for guiding retriever 718 along shaft 706 during insertion of scope 700 and withdrawal of shuttle 704. Fasteners 725 can be configured to release retriever 718 upon withdrawal of shuttle 704, such as by the proximal movement of retriever 718 and/or shuttle 704. In examples, fasteners 725 can comprise bands configured to attach to shaft 706 at one location and open or burst at another location to permit retriever 718 to be pulled proximally. In examples, fasteners 725 can be made of biocompatible material or bioresorbable material such that upon opening or bursting, fasteners 725 can be released from shaft 706 and absorbed into the anatomy over time in a safe manner.

FIG. 15B is a schematic side view of tissue collector 702 of FIG. 14A with shuttle 704 retracted from jaws 720A and 720B. As shown in FIG. 15B, shuttle 704 is shown pulled into portal 734 such that door 736 is shown pulled away from aperture 738. Additionally, retriever 718 is illustrated as being released from fastener 725. After jaws 720A and 720B have been operated to collect tissue sample 740, cage 723 can become impregnated or full of material so that it is difficult or impractical to obtain further tissue samples with jaws 720A and 720B. Thus, in order to increase the overall tissue-obtaining capability of scope 700 without multiple insertions of scope 700, shuttle 704 can be withdrawn from space 721 between jaws 720A and 720B to allow jaws 720A and 720B to collect additional tissue samples.

Retriever 718 can be pulled proximally from a proximal end portion of retriever 718 located outside of the patient, such as near handle section 32 (FIG. 1). Proximal movement of retriever 718 can pull cage 723 into engagement with door 736 to cause door 736 to be pushed away from aperture 738. Retriever 718 can extend between door 736 and aperture 738. Door 736 or aperture 738 can comprise a notch or cut-out to receive retriever 718 to allow door 736 to close even (e.g., lie flush) with aperture 738. In examples, door 736 can comprise a piece of flexible material attached to jaw 720B at end 742A and free around a remainder of the perimeter of door 736 including end 742B. Door 736 can be resilient so that end 742B can return to engagement with aperture 738 after shuttle 704 passes therethrough such as to facilitate withdrawal of tissue collector 702 from anatomy. In additional examples, door 736 can comprise a rigid body connected at end 742A to jaw 720B via a spring-loaded hinge so as to be biased shut. In other examples, door 736 can be omitted altogether.

Proximal pulling of retriever 718 can cause fastener 723 to release retriever 718. Thus, fastener 723 can burst or open to free separated ends 744A and 744B and allow retriever 718 to pull away from base 722 and allow cage 723 to pass proximally past fastener 723. In additional examples, fasteners 725 can be omitted and retriever 718 can comprise a rigid body configured to closely conform to the shape of shaft 706 such that retriever 718 can be pushed into the anatomy as a unit with scope 700.

Wires 724A and 724B of cage 723 can compress to pass through aperture 738. That is, wires 724A and 724B can rotate at hinges 726A and 726B to cause a reduction in the height of cage 723 (between wires 724A and 724B as shown in FIG. 15B) and a corresponding elongation of cage 723 (between hinges 726A and 726B as shown in FIG. 15B) to allow cage 723 to pass through aperture 738. Hinges 726A and 726B can be spring-loaded to facilitate re-expansion of cage 723.

Wires 724A and 724B can include barbs 746 to facilitate retention of tissue sample 740 within space 728. Barbs 746 can be located on the inside of wires 724A and 724B facing space 728 to engage with tissue sample 740. Barbs 746 can comprise micro-hooks or fish-hooks to prevent tissue sample 740 from passing out of cage 723, but that are configured to not interfere with anatomy of the patient while shuttle 704 is being withdrawn from the patient. In examples, barbs 746 can be oriented in a radial direction toward the center of space 728.

FIG. 16 is a schematic side view of a tissue shuttle 750 suitable for use with scope 700 and tissue collector 702 of FIG. 13. Tissue shuttle 750 can comprise corkscrew 752 and shaft 754, which can be coupled to mount 756. Corkscrew 752 can be scaled-up or scaled-down from the size shown in FIG. 16 relative to the size of jaws 720A and 720B. Tissue collector 702 can function similarly as is described with reference to FIGS. 15A and 15B except tissue shuttle 704 is replaced with tissue shuttle 750.

Tissue shuttle 750 can be loosely stored between jaws 720A and 720B or attached to one of jaws 720A and 720B with adhesive or the like. In the illustrated example, tissue shuttle 750 can be mounted between jaws 720A and 720B using mount 756. Mount 756 can comprise horizontal mount 758 and rotational mount 760. Horizontal mount 758 can be fixedly attached to tissue collector 702, such as at hinge 730. Horizontal mount 758 can hold shuttle 750 in a fixed axial and radial position relative to the axis of base 722. Rotational mount 760 can hold shaft 754 of shuttle 750 in a rotational manner. Rotational mount 760 can comprise bearings to facilitate rotation. That is, rotational mount 760 can permit shaft 754 to rotate axially relative to base 722. Retriever 718 can extend proximally from shaft 754 and can be used to impart rotational motion to shuttle 750. Thus, as jaws 720A and 720B are operated to collect tissue samples 740A and 740B, retriever 718 can be rotated to cause rotation of corkscrew 752. Jaws 720A and 720B can push tissue samples 720A and 720B into engagement with corkscrew 752. Rotation of corkscrew 752 can cause tissue samples 740A and 740B to advance proximally into jaws 720A and 720B to make room for additional tissue samples. In examples, retriever 718 and corkscrew 752 can be configured similar to a drain auger comprising a tightly wound coil that facilitates flexibility, axial pushing and pulling and transmission of rotational forces, wherein a distal end of the tightly wound coil is arranged in a bulbous configuration to collect tissue. After corkscrew 752 is filled with one or more tissue samples, retriever 718 can be pulled proximally to withdraw tissue shuttle 750 through portal 734 and out of the anatomy, breaking coupling with mount 756 upon initial pulling.

In additional examples, tissue shuttle 750 can be mounted in different positions between jaws 720A and 720B. For example, corkscrew 752 can be located so that the distal (rightmost in FIG. 16) tip of corkscrew 752 is further in jaws 720A and 720B, e.g., closer to shaft 722. As such, jaws 720A and 720B can receive tissue separated from anatomy without first engaging corkscrew 752. Corkscrew 752 can then be subsequently activated to engage the collected tissue. In additional examples, corkscrew 752 can be connected to a moveable mount that can change the position of corkscrew 752 between jaws 720A and 720B. For example, horizontal mount 758 can be configured to translate in the horizontal direction relative to the orientation of FIG. 16 such that the tip of corkscrew 752 can be moved relative to the tips of jaws 720A and 720B. As such, horizontal mount 758 can comprise or can be mounted to a linear actuator and the like to provide translation of corkscrew 752 .

As discussed above with reference to FIG. 9, in additional examples, a tissue shuttle can be configured as a needle array or one or more skewers disposed within or alongside jaws 720A and 720B to facilitate retrieval of collected tissue. Similarly, corkscrew 752 can be replaced with a one or more horizontally oriented skewers, e.g., needles or spikes upon which tissue can be slid. The skewers can be linearly actuated as discussed herein. Additionally, the skewers can include barbs, such as those used on fish hooks, that allow tissue pieces to be slid axially in the proximal direction onto the skewers over the barbs, and the barbs then inhibit axial sliding of the tissue pieces in the distal direction to prevent the tissue pieces from separating from the skewers during retrieval of the shuttle including the skewers.

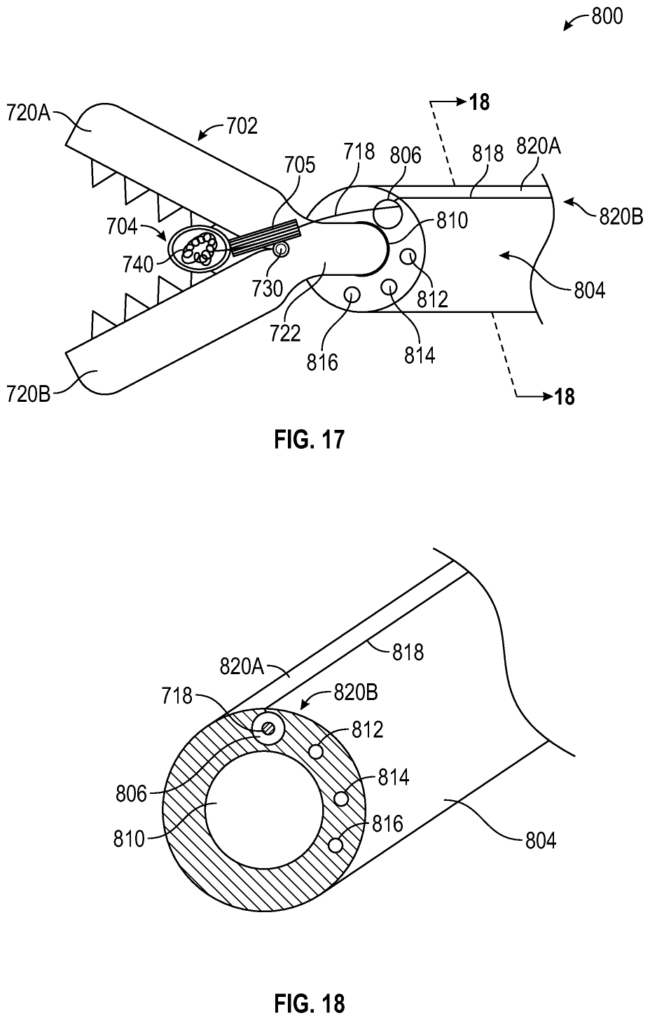

FIG. 17 is schematic perspective view of scope 800 and tissue collector 702. Scope 800 can comprise shaft 804 comprising openable lumen 806 configured to receive tissue shuttle 704. Shaft 804 can further comprise working lumen 810, imaging component 812, illumination component 814 and irrigation channel 816. Openable lumen 806 can comprise slit 818 forming retainer flanges, or flaps, 820A and 820B. Tissue shuttle 704 can be disposed within jaws 720A and 720B, as described with reference to FIG. 15A. Cage 723 can be filled with tissue sample 740 and retriever 718 can extend from cage 723 into openable lumen 806. As can be seen in FIG. 17, tissue shuttle 704 can be provided with projection 705. Projection 705 can be placed at the leading edge of shuttle 704 relative to proximal movement of shuttle 704 through openable lumen 806. Tether 718 can be connected to the proximal tip of projection 705. Projection 705 can comprise a guide body that is sized to fit within openable lumen 806. Thus, projection 705 can align shuttle 704 with the axis of openable lumen 806. Projection 705 can be sized smaller than openable lumen 806 to allow projection 705 to easily fit therein.

As discussed in greater detail with reference to FIGS. 18-20, openable lumen 806 can have a first, smaller cross-sectional area to allow retriever 718 to be positioned therein. However, in order to draw shuttle 704 proximally through shaft 804, without occupying excessive space outside of shat 804, lumen 806 can move distally through shaft 804 by pushing flanges 820A and 820B radially outward to increase openable lumen 806 to a second, larger cross-sectional area.

FIG. 18 is a schematic cross-sectional view of shaft 804 of FIG. 17 wherein openable lumen 806 is unobstructed by tissue shuttle 704 such that retainer flanges 820A and 820B are in an opposed state. Thus, openable lumen 806 is closed in a state that allows for easy insertion and withdrawal of shaft 804 through anatomy. Retainer flanges 820A and 820B can comprise flexible resilient portions of shaft 804 that can deflect outward under load or force (e.g., the position of FIG. 20), but that can return to their undeflected state (e.g., the position of FIG. 18) upon release of such load or force. Retainer flanges 820A and 820B can reduce or eliminate sharp edges that might interact with anatomy or other edges that might snag tissue to hamper the insertion process, such as in the case of having an uncovered channel extending along shaft 804. Furthermore, retainer flanges 820A and 820B can prevent openable lumen 806 from becoming obstructed with undesirable tissue or biological matter. Retainer flanges 820A and 820B can lay in opposition such that a small gap or space exists between the end faces of retainer flanges 820A and 820B. In other examples, retainer flanges 820A and 820B can contact or abut each other. As such, in a state of rest, retainer flanges 820A and 820B can be disposed in a state of rest, e.g., not flexed or not stretched, to close up or substantially close up (e.g., for a lumen encompassing approximately three-hundred-thirty degrees) openable lumen 806.

Jaws 720A and 720B can be operated to fill cage 723 with tissue sample 740. Once it is desired to withdraw shuttle 704 from tissue collector 702, retriever 718 can be pulled proximally to dislodge cage 723 from space 721 between jaws 720A and 720B. Shuttle 704 can be advanced proximally to engage the distal face of shaft 804.

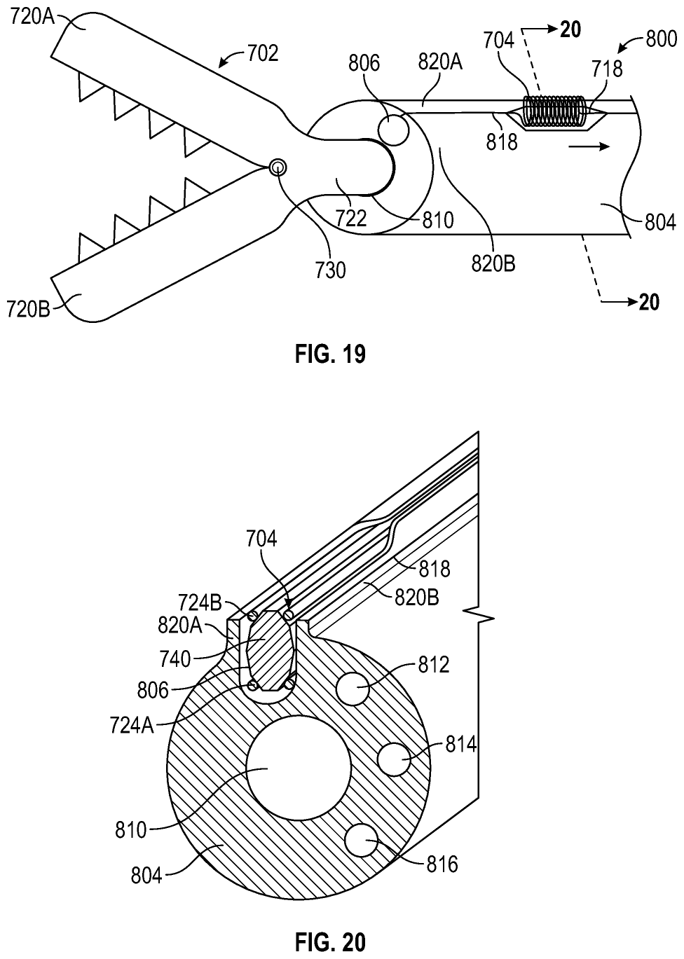

FIG. 19 is a schematic perspective view of shaft 804 of FIG. 17 wherein tissue shuttle 704 is located within openable lumen 806 such that retainer flanges 820A and 820B are turned out. FIG. 20 is a schematic cross-sectional view of shaft 804 of FIG. 19 wherein openable lumen 806 includes tissue shuttle 704 such that retainer flanges 820A and 820B are turned out. As can be seen in FIG. 19, flanges 820A and 820B can be rotated radially outward relative to the center axis of shaft 804. Flanges 820A and 820B can be configured such that flanges 820A and 820B are deflected only where, or in close proximity to where cage 723 is located. Thus, flanges 820A and 820B near the distal end face of shaft 804 and proximal of cage 723 can be in the relaxed state of FIGS. 17 and 18. As can be seen in FIG. 20, cage 723 of tissue shuttle 704 can be located between flanges 820A and 820B. Wires 724A and 724B, and other wires, of cage 723 can extend generally in the direction of the center axis of shaft 804 to slide against flanges 820A and 820B to facilitate siding in the proximal direction by retriever 718. The cross-sectional area of tissue sample 740 can be larger than the available cross-sectional area of openable lumen 806 with flanges 820A and 820B in a closed state. Thus, the cross-sectional area of openable lumen 806 can be reduced with flanges 820A and 820B closed to minimize the footprint of openable lumen 806 on the cross-sectional area of shaft 804. However, lumen 806 can open to allow tissue shuttle 704 that is engorged with tissue sample 740 to pass therethrough. After cage 723 is removed from shaft 804, lumen 806 can return to the reduced footprint size when shaft 804 is withdrawn from the anatomy.

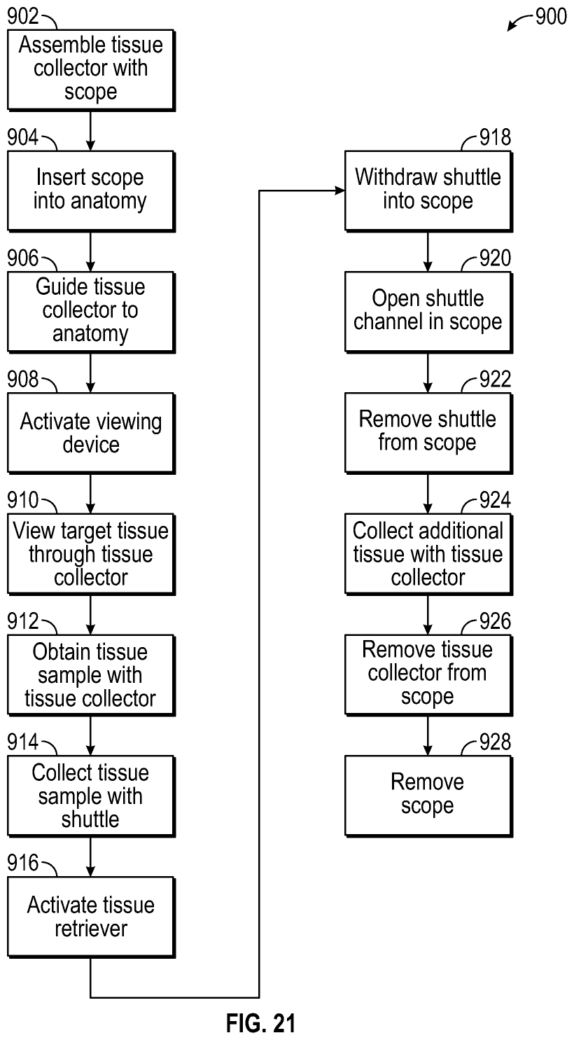

FIG. 21 is a block diagram illustrating methods 900 of collecting biological matter from anatomy of a patient using a tissue collection shuttle and an openable lumen. In examples, the tissue collection shuttle can be used without the openable lumen, and the openable lumen can be used without the tissue collection shuttle.

At step 902, a tissue collector can be assembled with a scope. In examples, the tissue collector can be tethered to the distal end of the scope, as discussed with reference to FIGS. 8A and 8B. In examples, the tissue collector can be inserted into the scope, as discussed with reference to FIGS. 5A and 5B. In examples, tissue collector 702 (FIG. 13) can be assembled with endoscope 402 (FIG. 8A) via tethering. In examples, tissue collector 702 (FIG. 13) can be assembled with endoscope 800 (FIGS. 17-20) via tethering. In additional examples, the expandable tissue retrieval devices, e.g., biopsy devices 500 and biopsy instrument 550 of FIGS. 10 and 11, respectively, can be assembled with scope 800 of FIGS. 17-20.

At step 904, the scope can be inserted into anatomy. In examples, the scopes discussed in step 902 can be inserted into a gastrointestinal tract (e.g., esophagus, stomach, duodenum, pancreaticobiliary duct, intestines, colon, and the like), renal area (e.g., kidney(s), ureter, bladder, urethra), other internal organs (e.g., reproductive systems, sinus cavities, submucosal regions, respiratory tract), and the like. In examples, the scope can be inserted into duodenum D and common bile duct 124 of FIG. 4. The scopes can be employed to collect tissue samples for various purposes, including removal of diseased tissue and biopsies.

At step 906, the tissue collector can be extended from the scope to reach anatomy where target tissue is located. The tissue collector can be operated, e.g., pushed, steered and guided, from the proximal end, such as at controller 206 (FIG. 5B) or handpiece 418 (FIG. 8A).

At step 908, a viewing device of the scope can be activated. For example, imaging unit 110 (FIG. 4) can be activated so that camera lens 137 can obtain images of tissue for transmission back to controller 108 and output unit 18 (FIG. 1). Likewise, lens 238 (FIG. 6A), lens 326 (FIG. 7A), imaging device 712 (FIG. 13) and imaging component 812 (FIG. 17) can be used to obtain images of the anatomy. The viewing device can be activated from controller 206 (FIG. 5B) or handpiece 418 (FIG. 8A). Images from the viewing device can be used to direct the tissue collector where to travel within the anatomy.

At step 910, target tissue can be viewed with the imaging device. Further, the target tissue can be viewed through the tissue collector. For example, imaging unit 110 can use lens 238 to display target tissue on output unit 18 (FIG. 1). Lens 238 can view the target tissue through transparent or translucent portions of the tissue collector, such as forceps 438 of FIG. 8B, biopsy instrument 450 of FIG. 9, biopsy instrument 500 of FIG. 10, biopsy instrument 550 of FIG. 11, and tissue collector 702 of FIG. 17. Light from a light source can be used to illuminate the target tissue. For example, light from lens 239, as generated by lighting unit 112, can be directed upon the target tissue. Additionally, light emitter 710 (FIG. 13) and illumination component 814 (FIG. 17) can be used to facilitate imaging of anatomy. As discussed herein, various components of the tissue collection devices can be configured to reflect light from the light source to enhance visibility.

At step 912, a tissue sample can be obtained with the tissue collector. A tissue separator, e.g., a forceps of the tissue collector can be navigated to the location of target tissue within the patient. For example, jaws 720A and 720B can be navigated through an anatomic duct to target tissue, such as target tissue 254 (FIG. 6A). The target tissue can comprise tissue that is potentially diseased or otherwise indicative of a diseased condition of the patient. Jaws 720A and 720B can be pushed, pressed or otherwise brought into pressurized contact with the target tissue. Thus, jaws 720A and 720B can be rotated about hinge 730 by activation of guide cables 414A and 414B (FIG. 8A) from handpiece 418 (FIG. 8A) to cause jaws 720A and 720B to slice, punch, shave, etc. one or more pieces of tissue away from the anatomy of the patient. As discussed in the present application, jaws 720A and 720B can be fabricated of clear, transparent or translucent material to facilitate passage of light from light emitter 710 and light to imaging device 712 to pass therethrough. Furthermore, portions of the tissue collection device can interact with light from any of the light emitting devices or components described herein to enhance visibility of such portions. For example, tissue separating components, such as teeth or blade edges, can be reflective or luminescent to enhance display on the video display monitor, such as output unit 18 (FIG. 1).

At step 914, tissue can be collected with the tissue collection shuttle. For example, tissue pulled into space 721 (FIG. 15A) by jaws 720A and 720B can pass into tissue shuttle 704, such as by passing between wires 724A and 724B. In examples, tissue shuttle 750 can be rotated via retriever 718 to allow tissue samples 740A and 740B to be gathered onto corkscrew 752.

At step 916, a retriever can be activated to withdraw the tissue collection shuttle. The retriever can be activated to release any fasteners, guides or couplings between the tissue collection shuttle and the tissue collector and the scope. For example, retriever 718 (FIG. 13) can be pulled proximally, such as by operating a lever, knob, spool, reel or the like at controller 206 (FIG. 5B) or handpiece 418 (FIG. 8A). Proximal movement of the retriever and the tissue shuttle can cause the tissue shuttle to break free from or release from fasteners or adhesive with jaws of a tissue separator, such as jaws 720A and 720B. Proximal movement of the retriever and the tissue shuttle can cause the retriever to break free from or release from fasteners or guides, such as fasteners 725, holding retriever in alignment with and engagement with the shaft of the scope.

At step 918, the tissue collection shuttle can be drawn into the shaft of the scope. In examples, the tissue collection shuttle can be drawn into a lumen extending along or within the shaft. In additional examples, the tissue collection shuttle can be pulled alongside the shaft of the scope, as shown in FIGS. 13-16. Tissue collection shuttle 704 (FIG. 13) can be pulled into openable lumen 806 (FIG. 17). Similarly, in examples, tissue collection shuttle 750 (FIG. 16) can be pulled into openable lumen 806.

At step 920, an openable channel or lumen on the shaft of the scope can open to accept the tissue collection shuttle. The openable channel or lumen can increase in cross-sectional area from a closed state to an open state. The lumen can be open to the exterior of the shaft to allow the tissue collection shuttle to be larger than the closed lumen. In examples, flanges 820A and 820B of openable channel 806 can deflect outward to allow the tissue shuttle to enter channel 806.

At step 922, the tissue collection shuttle can be removed from the scope. The tissue collection shuttle can be pulled through openable lumen 806 or another lumen to a proximal portion of the shaft of the scope that is located outside of the anatomy. In examples, the tissue collection shuttle can be pulled out of the anatomy alongside the shaft of the scope.

At step 924, additional tissue can be collected with the tissue collector by reapplying the tissue separator of the tissue collector. For example, jaws 720A and 720B can be operated to cut or otherwise separate a tissue sample from the target anatomy. As more tissue pieces are collected, the newly collected pieces can push the previously collected pieces further into the tissue retrieval device. The previously collected pieces can then activate the capacity enhancing features, such as by the previously collected pieces being pushed into engagement with sponge 454, being pushed into needle array 456, pushing movable jaws 504A and 504B outward, and moving deflectable wall 564.

At step 926, the tissue collector can be removed from the patient. In examples, the tissue collector can be removed from a daughter scope, and the daughter scope can be removed from a mother scope or duodenoscope, which can be left in place inside the anatomy. Safeguards can be put into place to ensure removal of the tissue collector without inadvertently cutting anatomy of the patient. The collected sample tissue can be removed from the tissue collection device. For example, jaws 720A and 7208B can be rotated away from each other to access space therebetween and remove sample tissue for analysis, etc.

At step 928, the scope can be removed from the patient. For example, scope 800 (FIGS. 17-20), scope 700 (FIG. 13) or endoscope 402 (FIG. 8A) can be withdrawn from the patient. The patient can thereafter be appropriately closed up or prepared for completion of the procedure.

### Various Notes & Examples

Example 1 is a tissue collection instrument comprising: a tissue separator device comprising: a separator; and a storage volume; and a tissue retrieval device comprising: a tissue shuttle configured to be storable in the storage volume; and a retriever connected to the tissue shuttle to retract the tissue shuttle away from the tissue separator device.

In Example 2, the subject matter of Example 1 optionally includes the tissue separator being at least partially transparent.

In Example 3, the subject matter of any one or more of Examples 1-2 optionally includes an endoscope comprising: an elongate shaft having a first lumen from in which the tissue separator device can be disposed; and a viewing device configured to view the tissue separator device distal of the first lumen.

In Example 4, the subject matter of Example 3 optionally includes the separator being at least partially made of a material capable of transmitting light.

In Example 5, the subject matter of any one or more of Examples 3-4 optionally includes the retriever comprising an elongate body extending with the elongate shaft.

In Example 6, the subject matter of Example 5 optionally includes the retriever comprising a rigid rod.

In Example 7, the subject matter of any one or more of Examples 5-6 optionally includes the retriever comprising a flexible tether.

In Example 8, the subject matter of any one or more of Examples 5-7 optionally includes the retriever extending alongside of the elongate shaft.

In Example 9, the subject matter of any one or more of Examples 5-8 optionally includes the retriever extending through the elongate shaft.

In Example 10, the subject matter of Example 9 optionally includes the elongate shaft including an openable channel extending along the elongate shaft.

In Example 11, the subject matter of Example 10 optionally includes the openable channel comprising first and second flanges located in opposition, wherein the first and second flanges are configured to flex outward from the elongate shaft to accommodate the tissue shuttle.

In Example 12, the subject matter of any one or more of Examples 3-11 optionally includes guides to attach the retriever to the elongate shaft.

In Example 13, the subject matter of Example 12 optionally includes the guides being configured to break-away when the retriever is pulled.

In Example 14, the subject matter of any one or more of Examples 1-13 optionally includes the separator comprising forceps comprising first and second jaws; and the storage volume is located between the first and second jaws.

In Example 15, the subject matter of Example 14 optionally includes the tissue shuttle being mounted within the forceps.

In Example 16, the subject matter of any one or more of Examples 14-15 optionally includes the tissue shuttle comprising a cage structure comprising a plurality of elongate bars.

In Example 17, the subject matter of Example 16 optionally includes the plurality of elongate bars comprising flexible wires.

In Example 18, the subject matter of any one or more of Examples 16-17 optionally includes the plurality of elongate bars being connected at hinges.

In Example 19, the subject matter of any one or more of Examples 14-18 optionally includes the tissue shuttle comprising a corkscrew and the storage volume comprises space around the corkscrew.

In Example 20, the subject matter of Example 19 optionally includes the corkscrew being rotatable by the retriever.

In Example 21, the subject matter of any one or more of Examples 14-20 optionally include wherein the forceps include a capacity increasing feature configured to increase an internal volume of at least one of the first and second jaws.

In Example 22, the subject matter of Example 21 optionally includes the capacity increasing feature comprising at least one of the first jaw and the second jaw being configured to translate in a direction opposite the other of the first and second jaws at a hinge to increase the space between the first and second jaws.

In Example 23, the subject matter of any one or more of Examples 21-22 optionally includes the capacity increasing feature comprising at least one of the first jaw and the second jaw being configured with a flexible wall to increase the space between the first and second jaws.

Example 24 is a method of collecting biological matter using a tissue collection instrument, the method comprising: inserting a tissue separator device into anatomy; operating the tissue separator device to obtain a first tissue sample from the anatomy; collecting the first tissue sample with a shuttle associated with the tissue separator device; and withdrawing the shuttle from the anatomy.

In Example 25, the subject matter of Example 24 optionally includes operating the tissue separator device to obtain a second tissue sample from the anatomy; and withdrawing the tissue separator device from the anatomy.

In Example 26, the subject matter of any one or more of Examples 24-25 optionally includes wherein inserting the tissue separator device into anatomy comprises: coupling the tissue separator device to a scope; and inserting the scope into the anatomy to deliver the tissue separator device to a site of target tissue.

In Example 27, the subject matter of Example 26 optionally includes viewing the site of the target tissue through transparent material of the tissue separator device with an imaging component of the scope.

In Example 28, the subject matter of any one or more of Examples 26-27 optionally includes collecting the first tissue sample with the shuttle associated with the tissue separator device by pulling a retriever attached to the shuttle.

In Example 29, the subject matter of Example 28 optionally includes pulling the retriever by pulling the retriever and the shuttle alongside a shaft of the scope.

In Example 30, the subject matter of Example 29 optionally includes pulling the retriever further by releasing the tether from a guiding device.

In Example 31, the subject matter of any one or more of Examples 28-30 optionally includes pulling the retriever comprises pulling the retriever and the shuttle through a lumen of the scope.

In Example 32, the subject matter of Example 31 optionally includes pulling the retriever through the lumen by opening a slot extending along the shaft with the shuttle as the shuttle moves through the shaft.

In Example 33, the subject matter of any one or more of Examples 24-32 optionally include collecting the first tissue sample with a shuttle associated with the tissue separator device by pushing the first tissue sample into a cage.

In Example 34, the subject matter of Example 33 optionally includes pushing the first tissue sample into a cage by flexing wires of the flexible cage.

In Example 35, the subject matter of any one or more of Examples 24-34 optionally includes collecting the first tissue sample with a shuttle associated with the tissue separator device by collecting the first tissue sample with a corkscrew.

In Example 36, the subject matter of Example 35 optionally includes collecting the first tissue sample with the corkscrew by rotating the corkscrew.

In Example 37, the subject matter of any one or more of Examples 35-36 optionally includes withdrawing the shuttle from the anatomy by passing the shuttle through a portal in the tissue separator.

In Example 38, the subject matter of any one or more of Examples 35-37 optionally includes passing the shuttle through a portal by pulling the shuttle through a flexible door.

In Example 39, the subject matter of any one or more of Examples 35-38 optionally includes passing the shuttle through a portal by disengaging the shuttle from a rotatable mount.

In Example 40, the subject matter of any one or more of Examples 24-39 optionally includes operating the tissue separator device to obtain the first tissue sample from the anatomy by operating forceps to sever a portion of the anatomy.

In Example 41, the subject matter of Example 40 optionally includes operating the tissue separator device to obtain the first tissue sample from the anatomy comprises increasing a capacity of the forceps with a capacity increasing feature.

In Example 42, the subject matter of Example 41 optionally includes increasing a capacity of the forceps with a capacity increasing feature by at least one of sliding a jaw of the forceps away from a pivot point to increase a distance from an opposing jaw and flexing a wall of a jaw of the forceps to increase an internal volume of the jaw.

Example 43 is a working shaft of a medical scope device, the working shaft comprising: an elongate body extending from a first end portion to a second end portion; an imaging component connected to the elongate body; a working channel extending at least partially through the elongate body between the first end portion and the second end portion; and a tissue retrieval channel extending at least partially through the elongate body between the first end portion and the second end portion, the tissue retrieval channel comprising an enlargeable cross-sectional area openable to an exterior of the elongate body.

In Example 44, the subject matter of Example 43 optionally includes the enlargeable cross-sectional area being non-stretchable.

In Example 45, the subject matter of any one or more of Examples 43-44 optionally includes the tissue retrieval channel comprising a pair of opposing flanges.

In Example 46, the subject matter of Example 45 optionally includes the pair of opposing flanges being deflectable away from a center axis of the tissue retrieval channel.

In Example 47, the subject matter of any one or more of Examples 45-46 optionally includes each opposing flange of the pair of opposing flanges being configured to abut each other in a state of rest.

In Example 48, the subject matter of any one or more of Examples 43-47 optionally includes the working channel being larger than the tissue retrieval channel.

Example 49 is a method of collecting biological matter comprising: assembling a tissue separator device with a scope; inserting the scope and the tissue separator device into anatomy; operating the tissue separator device to obtain a first tissue sample from the anatomy; and withdrawing the first tissue sample through an openable lumen in the scope.

In Example 50, the subject matter of Example 49 optionally includes withdrawing the first tissue sample through the openable lumen in the scope by withdrawing the tissue separator device through the openable lumen.

In Example 51, the subject matter of Example 50 optionally includes expanding the tissue separator device with one or more collected tissue samples.

In Example 52, the subject matter of any one or more of Examples 49-51 optionally includes withdrawing the first tissue sample through the openable lumen in the scope by withdrawing a tissue collection shuttle through the openable lumen.

In Example 53, the subject matter of Example 52 optionally includes pulling a retriever connected to the tissue collection shuttle to dislodge the tissue collection shuttle from the tissue separator device to enter the openable lumen.

Each of these non-limiting examples can stand on its own, or can be combined in various permutations or combinations with one or more of the other examples.

The above detailed description includes references to the accompanying drawings, which form a part of the detailed description. The drawings show, by way of illustration, specific embodiments in which the invention can be practiced. These embodiments are also referred to herein as “examples.” Such examples can include elements in addition to those shown or described. However, the present inventor also contemplates examples in which only those elements shown or described are provided. Moreover, the present inventor also contemplates examples using any combination or permutation of those elements shown or described (or one or more aspects thereof), either with respect to a particular example (or one or more aspects thereof), or with respect to other examples (or one or more aspects thereof) shown or described herein.

In the event of inconsistent usages between this document and any documents so incorporated by reference, the usage in this document controls.

In this document, the terms “a” or “an” are used, as is common in patent documents, to include one or more than one, independent of any other instances or usages of “at least one” or “one or more.” In this document, the term “or” is used to refer to a nonexclusive or, such that “A or B” includes “A but not B,” “B but not A,” and “A and B,” unless otherwise indicated. In this document, the terms “including” and “in which” are used as the plain-English equivalents of the respective terms “comprising” and “wherein.” Also, in the following claims, the terms “including” and “comprising” are open-ended, that is, a system, device, article, composition, formulation, or process that includes elements in addition to those listed after such a term in a claim are still deemed to fall within the scope of that claim. Moreover, in the following claims, the terms “first,” “second,” and “third,” etc. are used merely as labels, and are not intended to impose numerical requirements on their objects.

Method examples described herein can be machine or computer-implemented at least in part. Some examples can include a computer-readable medium or machine-readable medium encoded with instructions operable to configure an electronic device to perform methods as described in the above examples. An implementation of such methods can include code, such as microcode, assembly language code, a higher-level language code, or the like. Such code can include computer readable instructions for performing various methods. The code may form portions of computer program products. Further, in an example, the code can be tangibly stored on one or more volatile, non-transitory, or non-volatile tangible computer-readable media, such as during execution or at other times. Examples of these tangible computer-readable media can include, but are not limited to, hard disks, removable magnetic disks, removable optical disks (e.g., compact disks and digital video disks), magnetic cassettes, memory cards or sticks, random access memories (RAMs), read only memories (ROMs), and the like.

The above description is intended to be illustrative, and not restrictive. For example, the above-described examples (or one or more aspects thereof) may be used in combination with each other. Other embodiments can be used, such as by one of ordinary skill in the art upon reviewing the above description. The Abstract is provided to allow the reader to quickly ascertain the nature of the technical disclosure. It is submitted with the understanding that it will not be used to interpret or limit the scope or meaning of the claims. Also, in the above Detailed Description, various features may be grouped together to streamline the disclosure. This should not be interpreted as intending that an unclaimed disclosed feature is essential to any claim. Rather, inventive subject matter may lie in less than all features of a particular disclosed embodiment. Thus, the following claims are hereby incorporated into the Detailed Description as examples or embodiments, with each claim standing on its own as a separate embodiment, and it is contemplated that such embodiments can be combined with each other in various combinations or permutations. The scope of the invention should be determined with reference to the appended claims, along with the full scope of equivalents to which such claims are entitled.

1-53. (canceled)

54. A tissue collection instrument comprising:
a tissue separator device comprising:
an elongate shaft; and
a separator disposed at a distal end of the elongate shaft, the separator having a storage volume; and
a tissue retrieval device comprising:
a tissue shuttle configured to be storable in the storage volume; and
a retriever connected to the tissue shuttle and disposed radially outside of the elongate shaft to retract the tissue shuttle away from the tissue separator device.

55. The tissue collection instrument of claim 54, wherein the tissue separator device is at least partially transparent.

56. The tissue collection instrument of claim 54, further comprising an endoscope comprising:
an elongate working shaft having a first lumen from in which the tissue separator device can be disposed; and
a viewing device configured to view the tissue separator device distal of the first lumen.

57. The tissue collection instrument of claim 56, wherein the retriever includes an elongate body comprising a rigid rod or a flexible tether.

58. The tissue collection instrument of claim 57, wherein the retriever extends through the elongate working shaft.

59. The tissue collection instrument of claim 58, wherein the elongate shaft includes an openable channel extending along the elongate shaft.

60. The tissue collection instrument of claim 59, wherein the openable channel comprises first and second flanges located in opposition, wherein the first and second flanges are configured to flex outward from the elongate shaft to accommodate the tissue shuttle.

61. The tissue collection instrument of claim 57, wherein the retriever extends alongside of the elongate working shaft.

62. The tissue collection instrument of claim 61, further comprising guides to attach the retriever to the elongate working shaft.

63. The tissue collection instrument of claim 62, wherein the guides are configured to break-away when the retriever is pulled.

64. The tissue collection instrument of claim 54, wherein:
the separator comprises forceps comprising a first jaw and a second jaw; and
the storage volume is located between the first jaw and the second jaw;
wherein the tissue shuttle is mounted within the forceps.

65. The tissue collection instrument of claim 64, wherein the tissue shuttle comprises a cage structure comprising a plurality of elongate bars, wherein at least some of the plurality of elongate bars comprise flexible wires.

66. The tissue collection instrument of claim 64, wherein the tissue shuttle comprises a cage structure comprising a plurality of elongate bars, wherein at least some of the plurality of elongate bars are connected at hinges.

67. The tissue collection instrument of claim 64, wherein the tissue shuttle comprises a corkscrew and the storage volume comprises space around the corkscrew, wherein the corkscrew is rotatable by the retriever.

68. The tissue collection instrument of claim 64, wherein the forceps includes a capacity increasing feature configured to increase an internal volume of at least one of the first jaw and the second jaw, wherein the capacity increasing feature comprises at least one of the first jaw and the second jaw being configured to translate in a direction opposite the other of the first jaw and the second jaw at a hinge to increase space between the first jaw and second jaw.

69. The tissue collection instrument of claim 64, wherein the forceps include a capacity increasing feature configured to increase an internal volume of at least one of the first jaw and the second jaw, wherein the capacity increasing feature comprises at least one of the first jaw and the second jaw being configured with a flexible wall to increase space between the first jaw and the second jaw.

70. A method of collecting biological matter using a tissue collection instrument, the method comprising:
inserting a tissue separator device into anatomy;
operating the tissue separator device to obtain a first tissue sample from the anatomy;
collecting the first tissue sample with a shuttle associated with the tissue separator device by pulling a retriever attached to the shuttle; and
withdrawing the shuttle from the anatomy.

71. The method of claim 70, further comprising:
operating the tissue separator device to obtain a second tissue sample from the anatomy; and
withdrawing the tissue separator device from the anatomy.

72. The method of claim 70, wherein:
inserting the tissue separator device into anatomy comprises:
coupling the tissue separator device to a scope; and
inserting the scope into the anatomy to deliver the tissue separator device to a site of target tissue; and
operating the tissue separator device to obtain the first tissue sample from the anatomy comprises operating forceps to sever a portion of the anatomy.

73. The method of claim 72, wherein pulling the retriever comprises pulling the retriever and the shuttle alongside a shaft of the scope or pulling the retriever and the shuttle through a lumen of the scope.
